(19) United States
(12) Patent Application Publication
(10) Pub. No.: US 2026/0325703 A1
(43) Pub. Date: Oct. 22, 2026

(54) SURGICAL INSERT AND BONE CEMENT

(71) Applicants: UNIVERSITE D'ARTOIS, ARRAS (FR); UNIVERSITE DE LILLE, LILLE (FR); CENTRE HOSPITALIER UNIVERSITAIRE DE LILLE, LILLE (FR); INSTITUT NATIONAL DE LA SANTÉ ET DE LA RECHERCHE MÉDICALE, PARIS (FR); ECOLE NATIONALE SUPÉRIEURE MINES-TÉLÉCOM LILLE-DOUAI, VILLENEUVE D'ASCQ (FR); JUNIA, LILLE (FR)

(72) Inventors: Francine MONCHAU, RICHEBOURG (FR); Olivier CARPENTIER, LA CHAPELLE DE BRAIN (FR); Benoit GENESTIE, BETHUNE (FR); Nicolas BLANCHEMAIN, MERVILLE (FR); Gwenaël RAOUL, LILLE (FR)

(73) Assignees: UNIVERSITE D'ARTOIS, ARRAS (FR); UNIVERSITE DE LILLE, LILLE (FR); CENTRE HOSPITALIER UNIVERSITAIRE DE LILLE, LILLE (FR); INSTITUT NATIONAL DE LA SANTÉ ET DE LA RECHERCHE MÉDICALE, PARIS (FR); ECOLE NATIONALE SUPÉRIEURE MINES-TÉLÉCOM LILLE-DOUAI, VILLENEUVE D'ASCQ (FR); JUNIA, LILLE (FR)

(21) Appl. No.: 19/107,621

(22) PCT Filed: Aug. 31, 2023
(86) PCT No.: PCT/FR2023/051318

§ 371 Date: Feb. 28, 2025

(30) Foreign Application Priority Data
Sep. 1, 2022 (FR) 2208793
Sep. 1, 2022 (FR) 2208795

(51) Int. Cl. A61L 24/04; A61F 2/30; A61L 24/00; A61L 24/06; A61L 24/08

(52) U.S. Cl. CPC A61L 24/043; A61F 2/30734; A61L 24/001; A61L 24/06; A61L 24/08; A61F 2002/30011; A61F 2002/3013; A61F 2002/30593; A61F 2002/30784; A61F 2002/3092

(57) ABSTRACT

A surgical insert comprising a first layer and a second layer, the first layer and the second layer include openings having a figure-eight shape. A bone cement including a pulverulent solid phase with polysaccharides that are natural or derived from natural compounds and a ceramic filler, a liquid acrylate and a polymerizer.

### FIELD

The present disclosure relates to a surgical insert and a bone cement, in particular a surgical insert comprising a first layer and a second layer comprising figure-eight shaped openings and also a bone cement comprising a pulverulent solid phase comprising polysaccharides that are natural or derived from natural compounds and a ceramic filler and a liquid acrylate.

### BACKGROUND

Maxillofacial traumas are frequent traumas which mostly affect men and youth between 18 and 25 years old. They are mainly due to highway accidents, brawls, accidents related to playing sports, and domestic accidents.

In maxillofacial traumas, the main zones affected are teeth, and bones of the nose and orbital structure, which account for over 40% of cases because of their exposed position and very thin bone structure. A fracture of the orbital structure may lead to functional consequences. Thus, a trapping of muscle and/or fat which surrounds the eye may appear, causing a difference in elevation between the two eyes (diplody). It may cause compression of the infraorbital nerve (V₂) which passes under the orbital structure. This compression is responsible for difficulties with sensitivity in the zones around the eye like the cheek, nose, teeth and upper lip. It may also cause, in the case of a major fracture or collapse of the orbital structure, an enophthalmia, a dystrophy of the ocular globe. Finally, a fracture of the orbital structure may also have aesthetic repercussions when it is associated with a fracture of the orbital rim, nose or zygomatic body by changing the boney reliefs of the face.

In the case where the fracture of the orbital frame is sufficiently significant, it is necessary to reconstruct the orbital volume. This reconstruction must be rigorously respected because complications, like those mentioned above, may appear. In order for the ocular globe to recover its normal function and position, the reconstruction must be precise and conform to the anatomy of the orbit of the patient but also requires being accompanied by a repositioning of the intra-orbital soft tissue.

The advent of computer-assisted surgery has made many advances possible in the case of fractures requiring complex reconstructions of the bone volume. In particular it makes it possible to generate a virtual patient file in three dimensions faithfully reproducing the specific anatomical bone structure of the patient. It is thus possible to have available a faithful base for the reconstruction. It is particularly suited for the reconstruction of the orbital structure, a particularly complex zone of the human body and very different between individuals.

The main objective of reconstruction of the orbital structure is reconstituting the shape and volume of the orbit specific to the anatomy of each patient. However, the reduction of thin bone fragments of the orbital floor is sometimes not sufficient, or even possible. It is therefore necessary to cover or fill the defect to avoid complications like movement of tissues around the eye. Thus, the insertion of a biocompatible material, also called orbital implant, is sometimes indicated depending on the size and location of the defect in order to support the tissue around the eye and for remodeling the orbit. This orbital implant must however be adapted to the anatomy of each patient in order to function optimally.

Many materials of various characteristics may be used for fashioning implants for the repair of orbital lesions. The specifications for a material used as bone substitute include: biological, chemical and mechanical compatibility, osteoconductivity, the ability to allow different cellular elements to reach the bone area to be repaired, mechanical strength which must be near that of the bone, and macroporosity in order to allow the colonization of cells and the revascularization of the graft.

A first material used is autologous bone. Autologous bone is collected bone matter coming from another part of the body like for example the skull cap, the bone of the cranial vault, the iliac crest, one of the bones of the pelvis, or the microvascularized bones in case of extended reconstruction. This material is used for the reconstruction and augmentation of the contours of the orbit, in particular after resection of a tumor by a treatment based on ionizing radiation. However, the bone parts may not have an anatomically correct shape and a reduction of the bone volume by bone resorption appears. It is also possible to use chondrocostal cartilage.

A second material which may be used is based on resorbable polymer membranes. It is perfectly suitable for small defects, with a surface area below 1 cm². However, for larger defects, the resorbable polymer membranes do not have sufficient mechanical properties for supporting and stabilizing the orbital walls.

A third material commonly used is titanium and its alloys. It has been used for about 15 years and shows an excellent biocompatibility and an excellent integration in the bone site. Titanium is a material that has an excellent corrosion resistance, which is a useful property since the human body is particularly corrosive, and it has a high mechanical strength. It is easily shaped which makes it usable in many types of implants.

Finally, bioceramics are a fourth material used. They come in the form of macroporous granules or cylinders. They adapt to the defect to be filled and have good results in terms of bioactivity and osteoconduction. They however have the defect of needing to be shaped before implantation thereof.

A therapeutic solution to the bioceramics are bone substitutes in the form of cements. Cement is less invasive, practical to handle and adapts easily to the volume to be treated. There are two large families of cements. The first family is that of calcium phosphate cements. They are principally calcium phosphate compounds which give them many advantages like biocompatibility, bioresorption properties and bone regrowth. However, they have the disadvantage of being fragile and brittle. The second family of cements is represented by acrylic cements. They are principally made up of methyl polymethacrylate, formed by polyaddition and used for attaching prostheses. They have a good biocompatibility and also an excellent mechanical strength. However, they have the defect of having a behavior of shrinking after setting in situ leading to a gap between the implant and the bone. This behavior can lead to serious problems like the unsealing of some prostheses.

Further, beyond the characteristics from the specifications for a material usable as bone substitute, it appears that none of the materials previously cited has an excellent bioactivity with the medium in which it is implanted. In fact, a bioactivity and also an interaction of the material with the liquid medium forming the implant zone would in particular make it possible to use it as a matrix for the release of various substances, for example medications. It also appears that an implant with the capacity of expanding in volume by the absorption of fluids would be particularly suited to use as a bone implant by its capacity to get into close contact with the receiving bone site.

A first implant type is a manually shaped flat implant. In order to make it, the surgeon uses sterilized ABS (acrylonitrile butadiene styrene) replicas of the patient's skull for placing and modeling the implant in order to adapt it to the patient's anatomy. This implant nearly fully respects the patient's anatomy and thus improves the precision of the surgical repairs. However, this solution requires prerequisites: being able to fabricate and sterilize the patient's skull in ABS, desterilizing and resterilizing the implant, having the time to perform this operation and being in a health structure with access to this technology.

A second solution consists of designing implants for most individuals by anatomically preforming an implant covering the orbital floor and the median wall. These implants have “average” dimensions, meaning that their dimensions are not specific to one individual but are suitable to various anatomical shapes of the orbit. These implants are available in two different sizes. They offer a distinct improvement compared to standard implants. However, because fractures are not constant, in volume or location, and because the anatomical shape of the orbital wall is very complex, they do not perfectly cover the orbital wall.

Therefore, there is still a need for the design of an anatomically shaped orbital implant covering the seat of the lesion suited to each patient produced by rapid prototyping. The use of rapid prototyping will thus make it possible to optimize the shape of the implant and also optimize the internal structure thereof in order to allow a better circulation of fluids, bone recolonization and sufficient mechanical properties for support.

There is also a need to design the material filling the criteria of the specifications for material used as bone substitute but also that of the material capable of coming into close contact with the implant zone and being used as a matrix for release of substances.

### SUMMARY

The present disclosure aims to improve the situation.

According to a first aspect, a surgical implant is proposed comprising:
a first layer; and
a second layer,
   wherein the first layer and the second layer comprise openings having a figure-eight shape.

The surgical implant according to the first aspect of the invention has an internal architecture optimized for better circulation of fluids and bone regrowth. The first layer and the second layer make it possible for the surgical insert to have sufficient mechanical properties for support, in particular by a significant reduction of the maximum stresses. This surgical insert may be used as bone substitute, in particular human or in veterinary use. The general shape thereof may be adapted to result in an anatomically shaped bone implant. Thus, it could be used, for example, to replace a part of the skeleton like the orbital floor, serve as osteosynthesis plate, or form a joint prosthesis.

This surgical insert, or surgical implant, may further be used for human or animal surgery, in particular for vertebrates. It may be implanted or replace all or part of the structures of the human or animal body, for example a membrane, the skull, the trunk, etc. It may in particular be used in reparative surgery of the limbs (upper and lower), the face (including the jaws, teeth) or the trunk (for example the spinal column). It may also be used in vascular, abdominal or digestive surgery.

According to a second aspect, a bone substitute is proposed in bone cement form (also called bone cement) comprising:
a pulverulent solid phase comprising polysaccharides that are natural or derived from natural compounds and a ceramic filler;
a liquid acrylate;
a polymerizer; and/or
a radio-opacifier; and/or
a swelling-limiting agent.

The bone cement according to the second aspect of the invention has advantageous properties, in particular an excellent biocompatibility with the human body, an excellent osteoconductivity and a mechanical strength close to that of human bone. It also has a property of macroporosity allowing the colonization of the cells and the revascularization of the graft.

### BRIEF DESCRIPTION OF THE DRAWINGS

Other characteristics, details and advantages will appear upon reading the following detailed description, and analyzing the attached drawings, on which:

FIG. 1 shows schematically in top view a figure-eight shaped opening;

FIG. 2 shows schematically in top view figure-eight shaped openings arranged according to a triangular pattern;

FIG. 3 shows schematically in top view figure-eight shaped openings arranged according to a square pattern;

FIG. 4 shows schematically in top view figure-eight shaped openings arranged according to a hexagonal pattern;

FIG. 5 shows schematically in top view figure-eight shaped openings arranged according to a heterogeneous pattern;

FIG. 6 shows schematically in top view an example of a surgical insert according to the invention, with a square shape having attachment tabs by one of the sides thereof;

FIG. 7 shows schematically in top view an example of a surgical insert according to the invention with a round shape;

FIG. 8 shows schematically in top view an example of a surgical insert according to the invention, with a triangular shape having two zones of different porosities;

FIG. 9 shows the two faces of an example of a surgical insert according to the invention;

FIG. 10a shows schematically in top view a first layer of a surgical insert according to the invention;

FIG. 10b shows schematically in top view a second layer of the same surgical insert as in FIG. 10a;

FIG. 11 schematically shows an example of a surgical insert according to the invention, in three dimensions comprising two layers separated from each other by a plurality of spacers;

FIG. 12 schematically shows an example of a pillar with longitudinal axis that is straight and perpendicular to the middle plane of the first or second layer;

FIG. 13 shows the progression of the weighting coefficient C₂ as a function of the torsion angle β of the pillars of the surgical insert according to the invention;

FIG. 14 shows schematically an example of a pillar with vertical longitudinal axis of helical shape with a nonzero torsion angle;

FIG. 15 shows the progression of the weighting coefficient C₃ as a function of the inclination angle θ of the straight pillars of the surgical insert according to the invention;

FIG. 16 shows schematically an example of a pillar with a longitudinal axis having a nonzero inclination angle θ relative to the vertical;

FIG. 17 shows schematically an example of a pillar with a curved axis;

FIG. 18 shows schematically an example of a pillar with a straight longitudinal axis of helical shape with pores by the lateral surface thereof;

FIG. 19 shows schematically an example of a section of a layer of the surgical insert according to the invention and having a figure-eight shaped opening and figure-eight shaped transverse openings;

FIG. 20 shows schematically an example of a surgical insert according to the invention, in three dimensions having three layers separated by a plurality of pillars with a helical-shape and vertical longitudinal axis having pores at the lateral surface thereof;

FIG. 21 shows an example of a Bézier curve or function;

FIG. 22 shows a distribution of figure-eight shaped openings along a Bézier curve;

FIG. 23 is an enlargement of a part of the Bézier curve from FIG. 22 at one extremum;

FIG. 24 shows covering sections resulting from a homothetic duplication of the first layer arriving at the second layer;

FIG. 25 shows figure-eight openings arranged according to a partial rectangular pattern;

FIG. 26 shows figure-eight openings arranged partially along the contours of an octagon;

FIG. 27 shows an example of a surgical insert according to the invention and the bounding box thereof;

FIG. 28 shows the results of the simulation of two inserts both having figure-eight shaped openings, one having a mechanical reinforcement, and the other not;

FIG. 29 shows the ternary diagram of variation of cellulose acetate, ceramic filler and starch included in the bone cement according to the invention;

FIG. 30 shows the change of the Young's modulus in the domain of study of the bone cement according to the invention;

FIG. 31 shows the change of the shearing modulus in the domain of study of the bone cement according to the invention;

FIG. 32 shows the change of the swelling in the domain of study of the bone cement according to the invention;

FIG. 33 shows the change of temperature with time for three formulations of the bone cement according to the invention;

FIG. 34 shows the change of setting time of an acrylic cement as a function of the proportion by mass of vitamin C in the bone cement according to the invention;

FIG. 35 shows the change of maximum temperature reached by an acrylic cement as a function of the proportion by mass of vitamin C in the bone cement according to the invention;

FIG. 36 shows the change of the swelling of example 2.1 of the bone cement according to the invention in various liquid media as a function of the number of days of immersion;

FIG. 37 shows the change of the swelling of example 2.2 of the bone cement according to the invention in various liquid media as a function of the number of days of immersion;

FIG. 38 shows the change of the swelling of example 2.3 of the bone cement according to the invention in various liquid media as a function of the number of days of immersion;

FIG. 39 shows the change of the open porosity from examples 2.1, 2.2 and 2.3 of the bone cement according to the invention as a function of the number of days of immersion in distilled water;

FIG. 40 shows the evolution of the force of injectability for six botanical starch varieties;

FIG. 41 shows the cellular survival rate as a function of each formulation of the bone cement according to the invention and of two controls, a first based on 100% alumina, and the second based on tissue culture polystyrene (TCPS), material constituting the culture plates;

FIG. 42 shows the total quantities of ciprofloxacin (CFX) present in two different bone cement formulations according to the invention; and

FIG. 43 shows the CFX release profile with time for three formulations of the bone cement according to the invention.

### DETAILED DESCRIPTION

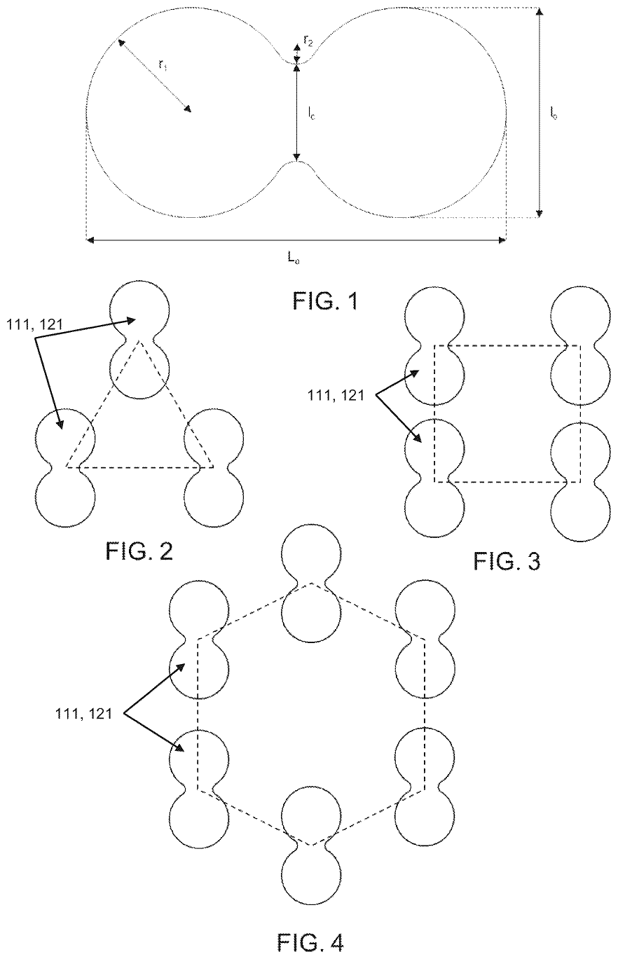

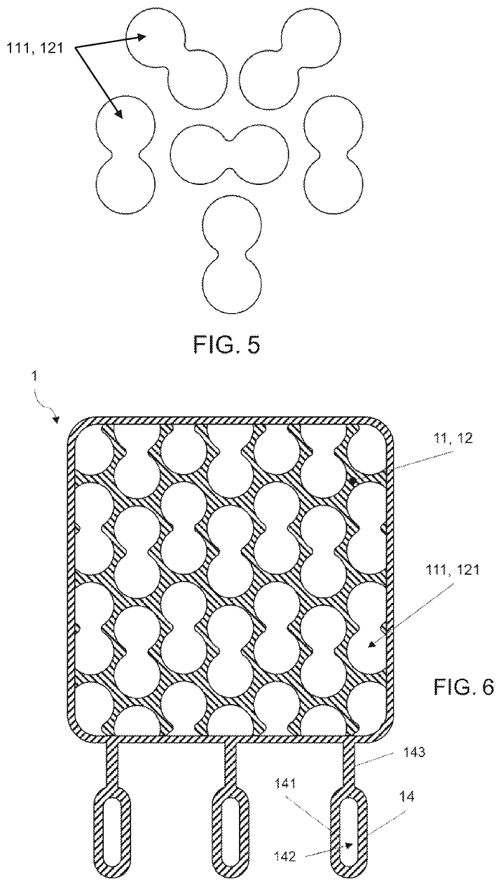

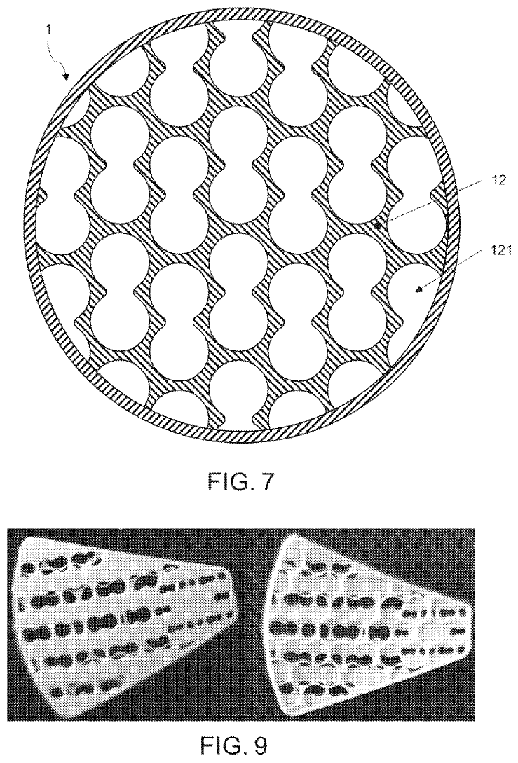

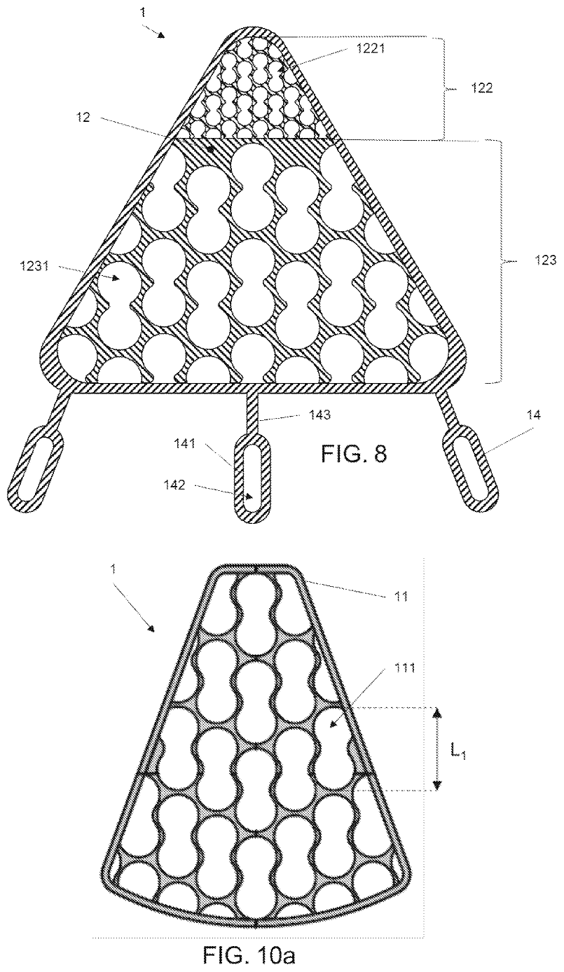

According to a first aspect, the present invention covers a surgical insert which will be described subsequently with reference to FIGS. 1 to 28.

The surgical insert 1 comprises:
a first layer 11; and
a second layer 12;
   wherein the first layer 11 and the second layer 12 comprise openings 111, 121 having a figure-eight shape.

Openings having the shape of the digit 8, meaning an elongated shape with two lobes at the end and a narrow central part, are described as “figure-eight shaped.”

The figure-eight shape may be described as composed of two end parts, each formed by a circle of radius r₁. The two end parts are connected to each other by a central part formed by a circular cove with radius r₂ such that the width of the central part is less than the width of the end parts, equal to 2×r₁. Thus, the radius r₁ is a first radius of curvature of the figure-eight shape in the end parts and the radius r₂ is a second radius of curvature of the figure-eight shape in the central part.

The figure-eight shape may also be described as a two-dimensional shape: oblong, tightened at the middle of the length thereof and comprising two end parts and a tightened central part. The figure-eight shape may have a curved contour, meaning that the contour does not have angles. The two end parts may have a first radius of curvature r₁ oriented towards the inside of the opening. The narrow central part may comprise a central zone having a second radius of curvature r₂ oriented towards the outside of the opening.

When it is indicated that an opening has a figure-eight shape, that means that a section of the opening has such a shape. The section generally considered is a section parallel to the median plane of the surface from which the opening extends.

An opening not having the figure-eight shape because of tiling and sizing of the insert will also be understood as “figure-eight shaped opening.” Meaning that any opening coming from a tiling by figure-eight shaped openings followed by a trimming to the desired shape of the insert is considered as a figure-eight opening.

The figure-eight shape may comprise a center of symmetry in the narrow central part. The figure-eight shape may have two axes of symmetry perpendicular to each other.

Several parameters may be defined to characterize the layers 11, 12 and the openings 111, 121:
r₁ the first radius of curvature;
r₂ the second radius of curvature;
L₀ the total length of the opening 111, 121;
[symbol] the total width of the opening 111, 121;
[symbol] the central width of the central part of the opening 111, 121;
h the thickness of the surgical insert 1;
L the length of the smallest side of the first and second layer 11, 12;
φ the porosity of a layer is the ratio between the surface area occupied by the openings 111, 121 of the layer to the total surface area of the layer 11, 12;
e the minimum distance between two openings 111, 121; and
F the force applied to the insert 1.

FIG. 1 shows a drawing in top view of a figure-eight shaped opening 111, 121. Thus, length is understood to be a dimension taken in a direction extending between the two lobes, the direction corresponding in particular to one of the axes of symmetry if present. Width is understood to be a dimension perpendicular to the length. The thickness of the surgical insert is understood as the distance between the two extreme faces of the insert in the direction of superposition of the layers. Unless otherwise indicated, the average of the thickness over the insert is understood. The distance between two openings is taken between the centers of these openings.

The total length, L₀, may be included between 200 μm and 50 mm. In some cases (for example for orbital floors), this length is preferably 200 μm to 2 mm. In other cases (for example for osteosynthesis plates), this length is preferably 10 to 50 mm.

The ratio of the total length, L₀, over the total width [symbol] , may be included between 1.8 and 2.2, preferably between 1.9 and 2.1, preferably between 1.95 and 2.05, for example 2.

The ratio between the first radius of curvature, r₁, and the second radius of curvature, r₂, may be included between 1.3 and 1.7, preferably between 1.4 and 1.6, preferably between 1.45 and 1.55, for example 1.5.

The ratio between the total width, [symbol] , and the central width [symbol] , may be included between 1.3 and 1.7, preferably between 1.4 and 1.6, preferably between 1.45 and 1.55, for example 1.5.

The preferred value of a parameter depends on the case studied and is generally a function of the maximum force which will be exerted on the insert 1 once in place and also of the general shape of the insert 1 which is going to change the distribution of the stress coming from loading.

The figure-eight shaped openings 111, 121 may be arranged at the layer 11, 12 according to a particular pattern. The pattern can be locally partial, meaning that some figure-eight shaped openings 111, 121 are missing. This pattern may be selected from a triangular pattern, a square pattern, a hexagonal pattern or a heterogeneous pattern. The pattern may further be selected such that the figure-eight shaped openings 111, 121 draw the contours of a geometric shape, in particular regular, like a triangle, rectangle, square, pentagon, hexagon, etc. A triangular pattern is a pattern in which the center of each opening 111, 121 is located at a vertex of a triangle. A square pattern is a pattern in which the center of each opening 111, 121 is located at a vertex of a square. A hexagonal pattern is a pattern in which the center of each opening 111, 121 is located at a vertex of a hexagon. A heterogeneous pattern is a pattern in which the center of each opening 111, 121 is arranged randomly. The distribution of openings according to a particular pattern does not predict the angular orientation of each of the openings around the center thereof. Thus, the orientation may be homogeneous (identical angular orientation for all the openings), centrifugal (different angular orientations for the opening such that the lengths thereof are collinear with a line connecting the center of the opening to the center of the pattern) or heterogeneous (at least two openings having different angular orientations).

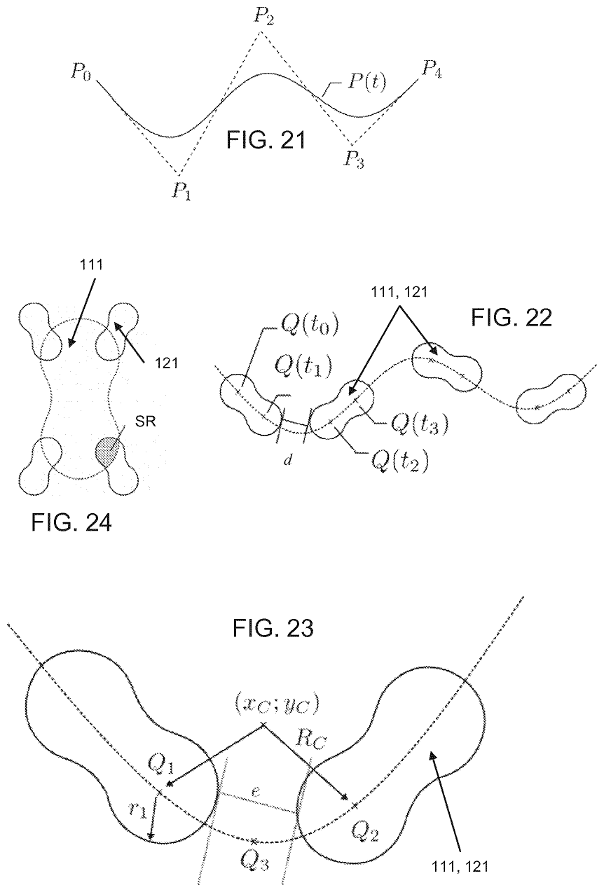

An example of a heterogeneous pattern may be described by using a Bézier curve or function (see FIG. 21) defined by a set of control points whose number is n and at least equal to 2, P₀, P₁, . . . , Pₙ₋₁. The following is the parametric representation:

$$P ( t ) = {\sum }_{j = 0}^{n - 1} {B}_{j}^{n - 1} ( t ) \times {P}_{j} ,$$

where the

$${B}_{j}^{n - 1}$$

our Bernstein polynomials:

$${B}_{j}^{n - 1} ( u ) = ( \begin{matrix}n - 1 \\ j\end{matrix} ) {{u}^{j} ( 1 - u )}^{n - 2} ,$$

$$where ( \begin{matrix}n - 1 \\ j\end{matrix} ) = {C}_{n - 1}^{j} = \frac{( n - 1 ) !}{j ! ( n - 1 - j ) !} .$$

In such a pattern, the center Q(t₀), Q(t₁), . . . of the circles of radius r₁ are placed on the Bézier curve (see FIG. 22). Preferably, a single figure-eight opening is placed between two consecutive extrema of the Bézier curve.

Further, the shortest measurable distance between the contours of two figure-eight shaped openings is greater than 0, preferably greater than 50 μm, also preferably greater than 100 μm and still preferably greater than 200 μm. Thus, the figure-eight shaped openings are disconnected.

Preferably, if n_{Q} is the total number of centers Q(t₀), Q(t₁), . . . of circles of radius r₁ and tᵢ is the parametric coefficient corresponding to the i^{th} center Q(tᵢ), then for i from 2 to n_{Q}, Q₁, Q₂ et Q₃ are defined as follows:

$${Q}_{1} = {\sum }_{j = 0}^{n - 1} {B}_{j}^{n - 1} ( {t}_{i - 1} ) \times {P}_{j} , is {the ( i - 1 )}^{t h} center ;$$

$${Q}_{2} = {\sum }_{j = 0}^{n - 1} {B}_{j}^{n - 1} ( {t}_{i} ) \times {P}_{j} , is the {i}^{t h} center ;$$

$${Q}_{3} = {\sum }_{j = 0}^{n - 1} {B}_{j}^{n - 1} ( \frac{{t}_{i - 1} + {t}_{i}}{2} ) \times {P}_{j} ,$$

is the point located between the (i−1)^{th} center and the i^{th} center.

A coordinate point (x_{C}, y_{C}) is also defined as follows:

$${x}_{C} = ( \frac{Δ {X}_{32}^{2} + Δ {Y}_{32}^{2}}{2 Δ {Y}_{32}} - \frac{Δ {X}_{21}^{2} + Δ {Y}_{21}^{2}}{2 Δ {Y}_{21}} ) \times {( \frac{Δ {X}_{21}}{Δ {Y}_{21}} - \frac{Δ {X}_{32}}{Δ {Y}_{32}} )}^{- 1} ;$$

$${y}_{C} = ( \frac{Δ {X}_{21}}{Δ {Y}_{21}} ) \times {x}_{c} + ( \frac{Δ {X}_{21}^{2} + Δ {Y}_{21}^{2}}{2 Δ {Y}_{21}} ) ;$$

$$and the following radius :$$

$${R}_{C} = \sqrt{{( Δ {X}_{1 C} )}^{2} + {( Δ {Y}_{1 C} )}^{2}} ;$$

$$where :$$

$$Δ {X}_{ab} = {x}_{a} - {x}_{b} ; Δ {Y}_{ab} = {y}_{a} - {y}_{b} ;$$

$$Δ {X}_{ab}^{2} = {x}_{a}^{2} - {x}_{b}^{2} ; Δ {Y}_{ab}^{2} = {y}_{a}^{2} - {y}_{b}^{2} ;$$

(xₖ, yₖ) are the coordinates of point Qₖ;
then, the placement of the centers of the circles of radius r₁ is preferably selected such that R_{C}≥γ×(2×r₁+d); where γ is a safety coefficient selected between 4 and 8, preferably 6 (see FIG. 23).
   This condition assures that the condition of minimal distance between two figure-eight shaped openings is satisfied.

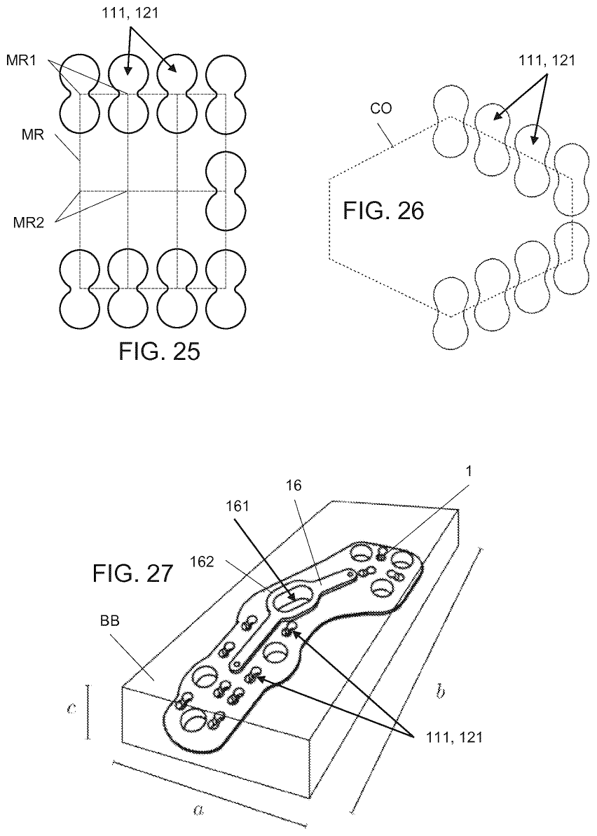

FIGS. 2, 3, 4 and 5 show respectively a top view drawing of figure-eight shaped openings arranged according to a triangular, square, hexagonal and heterogeneous pattern. In the case of the triangular pattern from FIG. 2, the square pattern from FIG. 3 and the hexagonal pattern from FIG. 4, the orientation of the openings is homogeneous. FIG. 25 shows a top view drawing of figure-eight shaped openings arranged according to a partial rectangular pattern. In FIG. 25, it may be seen that the figure-eight shaped openings are inserted in the nodes MR1 of a rectangular mesh MR but that other nodes MR2 of this mesh are not occupied by a figure-eight shaped opening. FIG. 26 shows a top view drawing of figure-eight shaped openings arranged partially along the contours of a hexagon. FIG. 22 shows a top view drawing of figure-eight shaped openings arranged according to a heterogeneous pattern described by a Bézier curve.

The porosity of the layers 11, 12 may be from 0.1 to 0.93, preferably from 0.4 to 0.8.

The two layers are preferably parallel to each other.

The porosity of a layer 11, 12 and also the mechanical strength of a layer 11, 12 are two values which change in opposite direction. When the porosity is included in the intervals defined above, the layer 11, 12 has an elastic-limit stress below 830 MPa which is the conventional elastic limit (Rp0.2) of the titanium implant TA6V.

The greater the porosity, the lighter the structure will be. Additionally, more the circulation of fluids will be improved. The mechanical strength of the insert 1 decreases with increasing porosity.

The figure-eight shaped openings 111, 121 may be arranged parallel at the layer 11, 12 and in at least one zone, meaning that the eights forming these openings 111, 121 are all oriented in the same direction in this zone. In some cases, all the openings 111, 121 of the layer 11, 12 are parallel to each other.

The surgical insert 1 may further comprise an attachment tab 14 extending from one of the layers. The attachment tab 14 may have an oblong shape attachment 141, in particular with an opening 142, also oblong, connected to the first and/or second layers 11, 12 by tongue 143.

The surgical insert 1 may have various shapes, for example a square, round or triangular shape and more generally any shape physiologically compatible with the reconstruction of a structure of the human body.

FIGS. 6, 7 and 8 show schematically in top view a surgical insert 1 respectively with a square shape having attachment tabs at one of the sides thereof, round shape and triangular shape. The triangular shape insert 1 is shown with two zones of different porosities.

The surgical insert 1 may have a circular section shape truncated on the apex side. Such as surgical insert is advantageous for the reconstruction of the floor of the orbit.

The surgical insert 1 may be made of a material selected among a polymer, metal, ceramic or composite, where these materials are medical grade.

When the insert 1 is made of a polymer, it may in particular be composed of polyetheretherketone, polylactic acid or derivatives thereof.

When the insert 1 is made of a metal, it may in particular be made of titanium, a titanium alloy, stainless steel, a cobalt alloy (such as cobalt chrome), or tantalum.

When the insert 1 is made of a ceramic, this ceramic may be a bioglass or bioceramic, in particular calcium phosphate bioceramic, for example hydroxyapatite, tricalcium phosphate (TCP), and mixtures thereof.

When the insert 1 is made of a composite, it may in particular be made of the bone cement described below in more detail.

The insert 1 may also be made of a bone cement, in particular the one described below.

Generally, the surgical insert 1 according to the invention may be made from a patient scan. For example, it may be made of a polymer, metal or bioceramic material before being covered with the bone cement described below or be constituted exclusively of the bone cement by rapid prototyping. The insert 1 is next washed for eliminating any residual traces; it is washed in ether then in water before being placed to dry in an oven at 37° C. for 24 hours. Have an active ingredient needs to be added, the insert 1 may be in contact with the medication before implantation for biofunctionalization thereof.

According to an embodiment, the ratio L/h of the length L of the shortest side of the first 11 and second layers 12 and the thickness h of the surgical insert 1 is selected larger than 20.

Also, the thickness h of each layer must satisfy the relationship:

$$h > \frac{min \{ l , L \}}{4} \qquad [ Math . 1 ]$$

where l is the maximum width of the surgical insert and L is the maximum length of the surgical insert.

In the following, we will describe the values of the parameters in the case where the ratio L/h is greater than 20.

The first radius of curvature may be greater than or equal to the minimum first radius of curvature of r_{1,min} 345 μm and may be less than or equal to the maximum first radius of curvature r_{1,max} defined by Math. 2.

$${r}_{1 , max} = \sqrt{\frac{S φ}{N}} \qquad [ Math . 2 ]$$

where Sis a total surface area of the insert 1, φ is the total porosity of the insert 1, and N is the number of figure-eight shaped pores in one layer.

When the first radius of curvature is included between the boundaries defined above, it makes a good osteogenesis possible, meaning that it makes possible the formation and development of bone where it is inserted. The lower boundary was determined by fluid mechanics; it agrees with the openings conventionally used in bioceramics. To determine the upper boundary, a parametric study was done on 190 cases by varying the geometric dimensions and the loads applied to the unit cell.

The minimum distance between two openings 111, 121 may be greater than or equal to 100 μm. The minimum distance between two openings 111, 121 may be less than or equal to four times the first radius of curvature. If two openings 111, 121 have different sizes, then the minimum distance between two openings 111, 121 may be less than or equal to four times the radius of curvature of the larger size opening 111, 121. The lower boundary sets the minimum load that the insert can bear. The interval makes it possible to assure the circulation of fluids.

The thickness h of the inset 1 may be greater than or equal to the minimum thickness hₘᵢₙ 100 μm and may be less than or equal to the maximum thickness defined by Math. 3.

$${h}_{max} \leq \frac{{r}_{1} + e}{10} \qquad [ Math . 3 ]$$

The only real maximum limit on the thickness of the insert 1 is the biological limit, meaning the size of the insert 1 must be suited to the zone in which it is inserted and to the trauma that it must repair. However, it may be advantageous that the insert 1 remains within the Kirchhoff-Love hypotheses. In which case, the maximum thickness must satisfy the relation Math. 3.

The allowable load is then defined by the relation Math. 4 for which the parameters are given in Table 1.

$$\vert {F}_{max} \vert \leq A . {e}^{- B φ} \cdot [ \frac{k}{f ( a )} φ eh ] \qquad [ Math . 4 ]$$

The maximum allowable load supported by the insert 1 may be determined by using the Kirchhoff-Love hypothesis and also by the geometric parameters of the insert 1 previously defined.

TABLE 1

| | | Square | Hexagonal |
|---|---|---|---|
| Parameter | Designation | pattern | pattern |
| A | Coefficient | 1.5 to 1.6 | 1.5 to 1.6 |
| B | Coefficient | 5 to 6 | 4 to 5 |
| k | Coefficient of | 16,000 | 13,000 |
| | allowable stress | to | to |
| | | 17,000 | 14,000 |
| f(α) | Parameter α | | |
| | correlation function | | |
| α | Angle between the | | |
| | opening and the | | |
| | vector of the load | | |
| | applied to the insert | | |

The relation between f(α) and α is given in Table 2 below:

TABLE 2

| α | 0 | 10 | 20 | 30 | 40 | 50 | 60 | 70 | 80 | 90 |
|---|---|---|---|---|---|---|---|---|---|---|
| f(α) | 1 | 1.18 | 1.30 | 1.39 | 1.42 | 1.54 | 1.62 | 1.63 | 1.64 | 1.66 |

These values allow the best compromise between sizes and repetitions of the openings 111, 121. These values make it possible to have a better circulation of fluids, a better bone recolonization and sufficient mechanical properties for support.

As it relates to nonuniform patterns of openings 111, 121, the person skilled in the art would know how to verify the mechanical behavior of the insert 1 by a final numerical analysis in order to confirm the proposed size and distribution of openings 111, 121.

The first layer 11 may have a greater porosity than the porosity of the second layer 12. The ratio of the maximum length of the openings 111 of the first layer 11 L₁ and of the maximum length of the first openings 121 of the second layer 12 L₂ may be greater than 1.4, preferably it is greater than 1.4, and less than 3.2.

The objective of the first layer 11 is to be directly in contact with the bone structure. Thus, preferably, the first layer 11 is a material biocompatible with osteoblasts for incorporating them in the bone tissue without risk of formation of fibrous tissues. The objective of the second layer 12 is to support the tissues or organs. Thus, preferably, it has a smooth and not very porous surface in order to avoid infections.

The second layer 12 may have an orientation guide.

A second layer 12 may have two zones, a first zone 122 with openings 1221 having a first maximum length L₂ and a second zone 123 with second openings 1231 having a second maximum length. The ratio between the first maximum length, L₃, and the second maximum length, L₂, may be included between 1.6 and 2, preferably between 1.7 and 1.9, preferably between 1.75 and 1.85, for example 1.8.

The ratio between the maximum length of the openings 111 of the first layer L₁ and the first maximum length L₂ may be included between 1.4 and 1.8, preferably between 1.5 and 1.7 preferably between 1.55 and 1.65, for example 1.6.

The ratio between the maximum length of the openings 111 of the first layer L₁, and the second maximum length, L₃, may be included between 2.8 and 3.2, preferably between 2.9 and 3.1, preferably between 2.95 and 3.05, for example 3.

The first zone 122 with a lower porosity serves to increase the stability of the insert 1. This denser zone 122 may also be useful for the surgeon when they place the insert 1 in the damaged zone. Indeed, the figure-eight shaped openings 121 with a smaller size may serve as a guide for knowing the direction for positioning the insert 1.

This two-layer structure allows fluids to easily move through the insert 1 in order to avoid any stagnation and/or risk of infection.

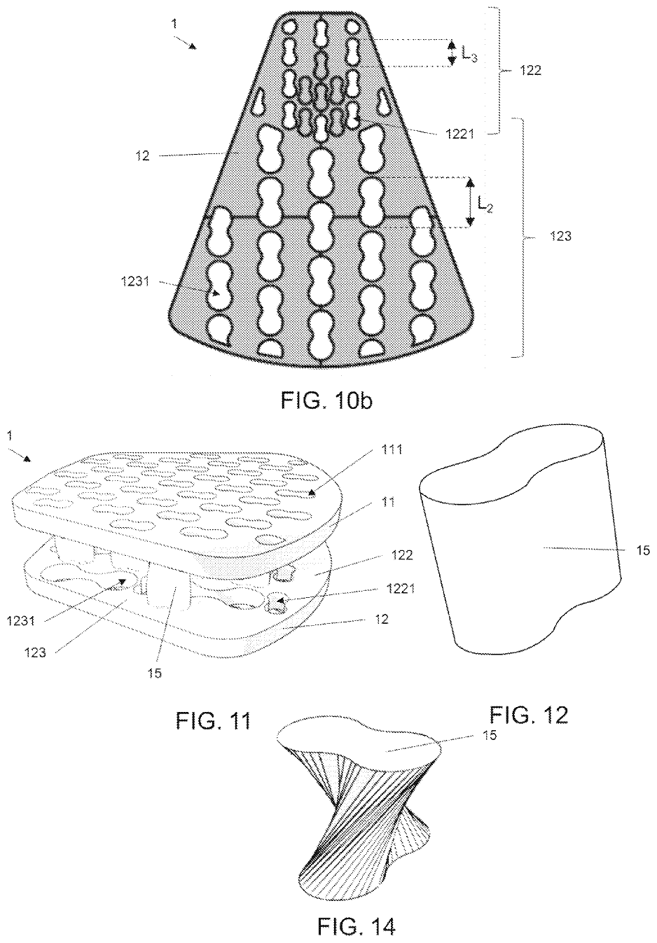

FIG. 9 shows the two faces of an example of such a two-layer structure. FIGS. 10a and 10b respectively show a top view of the first layer 11 and a top view of the second layer 12.

The total porosity of the insert 1 is calculated with the following formula:

$${φ}_{tot} = \frac{{φ}_{1} {S}_{1} + {φ}_{2} {S}_{2}}{{S}_{1} + {S}_{2}} \qquad [ Math . 5 ]$$

where φₜₒₜ is a total porosity of the surgical insert 1, φ₁ is the porosity of the first layer 11, φ₂ is the porosity of the second layer 12, S₁ is the total surface area of the first layer 11 and S₂ is a total surface area of the second layer 12. For a given layer, the porosity is the ratio between the surface area occupied by the figure-eight shaped openings and the total surface area of the layer.

Preferably, the insert 1 has a total porosity greater than or equal to 0.6 and less than or equal to 0.93. Preferably, the insert 1 has a ratio between the total surface area occupied by the openings of one layer and the total surface area of this layer greater than or equal to 0.09 and less than or equal to 0.92.

When the pattern of the openings is a hexagonal pattern, the ratio between the total surface area occupied by the openings of one layer and the total surface area of this layer is greater than or equal to 0.111 and less than or equal to 0.923.

According to another embodiment, the ratio L/h is less than 4. Subsequently, the characteristics will be described in the case of a ratio L/h less than 4. Further, the fluid flows are then generally complex.

A second layer 12 may have two zones, a first zone 122 with openings 1221 having a first maximum length L₂ and a second zone 123 with second openings 1231 having a second maximum length. The ratio between the first maximum length, L₃, and the second maximum length, L₂, may be included between 1.6 and 2, preferably between 1.7 and 1.9, preferably between 1.75 and 1.85, for example 1.8.

In the case of figure-eight shaped openings 111 distributed according to a heterogeneous pattern described by a Bézier curve on the first layer 11, the arrangement of the figure-eight shaped openings 121 on the second layer 12 may correspond to a homothetic duplication of the arrangement of the figure-eight shaped openings 111 on the first layer 11. This means that the openings undergo a translation and rotation (duplication) and then a homogeneous scaling (homothetic). This homothetic duplication may be described by the following formulas:

$${M}_{2} ( x , y , z ) = [ \begin{matrix}1 & 0 & 0 & X \\ 0 & 1 & 0 & Y \\ 0 & 0 & 1 & Z \\ 0 & 0 & 0 & 1\end{matrix} ] \times [ \begin{matrix}cos θ & - sin θ & 0 & 0 \\ sin θ & cos θ & 0 & 0 \\ 0 & 0 & 1 & 0 \\ 0 & 0 & 0 & 1\end{matrix} ] \times [ \begin{matrix}{s}_{E} & 0 & 0 & 0 \\ 0 & {s}_{E} & 0 & 0 \\ 0 & 0 & {s}_{E} & 0 \\ 0 & 0 & 0 & 1\end{matrix} ] {M}_{1} ( x , y , z ) ;$$

where:
M₁(x,y,z), the coordinates of the centers of the figure-eight shaped openings of the first layer;
M₂(x,y,z), the coordinates of the centers of the figure-eight shaped openings of the second layer;
X, the value of the translation along the main vector X;
Y, the value of the translation along the main vector Y;
Z, the value of the translation along the main vector Z;
θ, the angle of rotation about the main axis Z; and
s_{E}, the scaling coefficient.

There may be a single set of openings 121 of the second layer 12 or several sets of openings 121 of the second layer 12 distinguished from each other by the angle of rotation θ and/or the scaling coefficient s_{E}. Thus, from one opening 111 of the first layer 11, there may be one, two, three or four openings 121 of the second layer 12, or more.

Preferably, the homothetic duplication leads to a minimal section of overlap SR of 0.2 mm² between the opening 111 of the first layer 11 and the opening 121 of the second layer 12 resulting from it (see FIG. 24). Still preferably, the overlap amounts to at most one quarter of the surface area of the opening 111 of the first layer 11.

A plurality of spacers 15 may be placed between the first layer 11 and the second layer 12.

Each spacer 15 may have the shape of a pillar 15. FIG. 11 shows a surgical insert in three dimensions comprising two layers separated from each other by a plurality of pillars with figure-eight shaped section.

The pillar 15 may have a longitudinal axis. This longitudinal axis may be straight (FIGS. 12, 14 and 16) or curved (FIG. 17). When the longitudinal axis is straight, it may extend perpendicularly from the median plane formed by the first layer 11 or by the second layer 12. It may also be understood by forming a non-right angle with this median plane. When the longitudinal axis is curved, it follows the curve of a parametric equation between the first layer 11 and the second layer 12.

Each spacer 15 among the plurality of spacers 15 may have a transverse section which has a figure-eight shape as previously described. Thus, the pillar 15 may be obtained by translation of the figure-eight shape along the longitudinal axis (FIGS. 12, 16). Alternatively, the pillar 15 may be obtained by a combination of translation and rotation of the figure-eight shape along the longitudinal axis so as to confer to the lateral surface of the pillar 15 a helical shape (FIG. 14).

In the case of a pillar 15 with a helical-shaped lateral surface, preferably, the number of rotations of the figure-eight shape between the first layer 11 and the second layer 12 may be ¼+k, ½+k, ¾+k or 1+k where k is a digit greater than or equal to 0, for example 0, 1, 2, 3. In the case where the number of rotations is ¼+k or ¾+k, this means that the orientation of the figure-eight shape at the second layer 12 is) 90° (270° of its orientation at the first layer 11. In the case where the number of rotations is ½+k or 1+k, this means that the orientation of the figure-eight shape at the second layer 12 is 180° (360°) of its orientation at the first layer 11.

More generally, in the case where the pillar 15 is chosen as being a helically shaped pillar 15 with a torsion angle β, said angle β may be included between 0 and 180°, preferably less than 90°.

The parametric equation allowing design of the helically shaped pillar 15 is expressed below.

$$x ( t ) = r \times cos ( t ) , y ( t ) = r \times sin ( t ) , z ( t ) = \frac{5.440}{β} r \times t \qquad [ Math . 6 ]$$

where t and β are expressed in radians.

The spacers 15 are preferably sized for bearing the load and in particular in order to take up the normal force of compression and to resist buckling.

In order to study the influence of the various parameters of the pillars 15 on the allowable load, the following parameters are defined and the extreme values thereof will be determined by a parametric analysis:
H: the height of the pillar 15 measured as the distance between the first layer 11 and the second layer 12,
B: the torsion angle of the pillar 15; this angle is nonzero when the pillar 15 is helical,
θ: the angle of inclination of the pillar 15 for a straight pillar 15, and
C: the curve of a parametric equation for curved pillar 15.

Each pillar 15 among the plurality of pillars 15 may have a height greater than or equal to 3 mm. The height is considered perpendicular to the median plane of the first or second layer. The maximum value Hₘₐₓ of the height of the pillar 15 may satisfy the following formula:

$${H}_{max} = 637.040 \times {F}_{p}^{- 0.493} \times {r}_{1}^{2} \qquad [ Math . 7 ]$$

where F_{P} is the total force that the pillar 15 may support for a straight pillar 15, and r₁ is the first radius of curvature.

The helical pillar 15 may support an allowable load increased by a factor C₂ expressed by formula Math 8.

$${F}_{p}^{m} = {C}_{2} {F}_{p} , \{ \begin{matrix}{C}_{2} = 0.002093 β + 1.00042 , β \leq 90 ° \\ {C}_{2} = 0.007795 β + 0.404444 , β > 90 °\end{matrix} \} \qquad [ Math . 8 ]$$

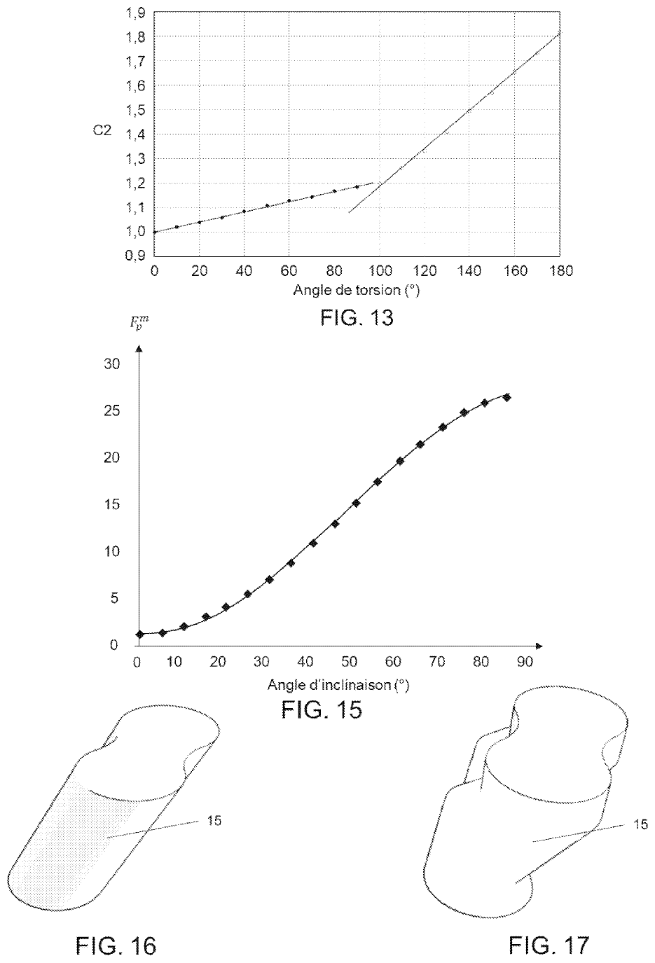

FIG. 13 shows the progression of the weighting coefficient as a function of the torsion angle β. This weighting coefficient may be determined by parametric analysis. Thus, it is observed that beyond 90°, the weighting coefficient C₂ greatly increases.

In the case where the pillar 15 is a selected as being a straight axis pillar 15 with an angle of inclination θ; said angle θ may be included between 0 and 85°. It is preferable to place a pillar 15 in mirror with an opposite angle so that the support is more effective. The angle of inclination is measured relative to the normal to the median plane formed by the first or second layer.

The straight axis pillar may support an allowable load increased by a factor C₃ expressed by the formula Math. 9.

$${F}_{p}^{m} = {C}_{3} {F}_{p} , {C}_{3} = 0.000 \; 068 {θ}^{3} + 9.7 \; 11 {θ}^{2} - 0.0 \; 386 θ + 1.1782 \qquad [ Math . 9 ]$$

FIG. 15 shows the progression of the weighting coefficient C₃ as a function of the inclination angle θ. This weighting coefficient may be determined by parametric analysis. It is observed that the larger the angle of inclination, the more the weighted force increases.

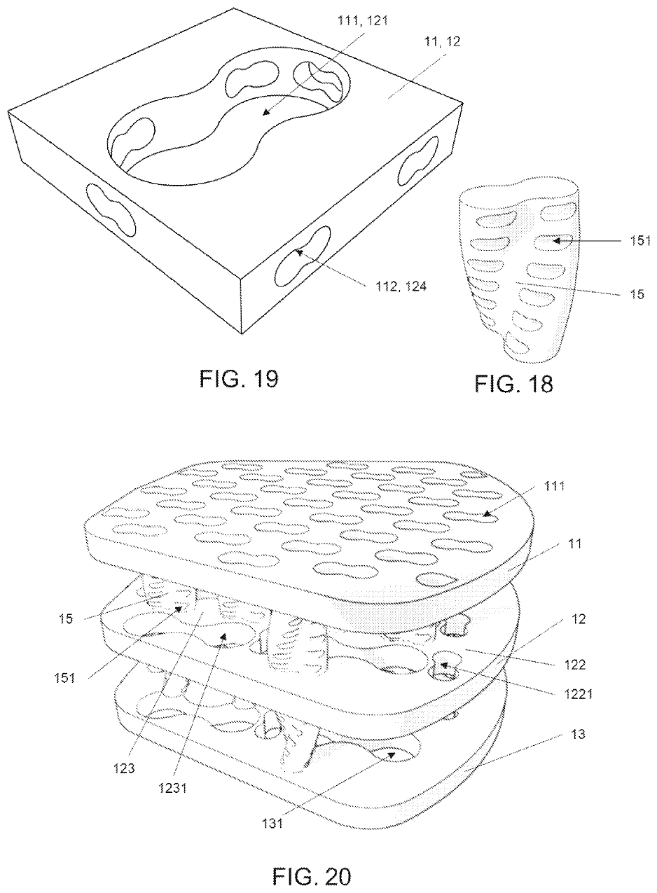

Each pillar 15 may comprise through openings 151 at the lateral surface thereof (FIG. 18). These through openings 151 serve to improve the circulation of fluid between the first layer 11 and the second layer 12. In fact, the presence of pillars 15 forces the fluids to move around them, the openings 151 thus allow a portion of the fluids to pass through the pillars 15, making the flow less complex.

The ratio between the surface area of the transverse openings 151 and the lateral surface area of the pillar 15 may be included between 0.25 and 0.3.

As shown in FIG. 19, the openings 111, 121 of the first layer and/or the second layer may have a lateral wall formed in the thickness of the corresponding layer, and in which the first layer 11 and/or the second layer 12 comprises transverse openings 112, 124 passing through figure-eight shaped sections such as previously described and extending between the lateral wall of two openings 111, 121 of the corresponding layer 11, 12. Alternatively, the transverse openings 112, 124 may have a circular or oval section. These transverse openings 112, 124 may be entirely in the thickness of the first layer 11 or the second layer 12 (i.e. the transverse openings 112, 124 form channels) or on the surface thereof (i.e. these transverse openings 112, 124 form channels with a free surface). The two types of channels may coexist in a single insert 1.

The number of transverse openings 112, 124 may be greater than or equal to 4 and less than or equal to 8, for example 4, 5, 6, 7 and 8.

In the case where, like in the insert 1 in three dimensions, the thickness of the insert 1 is of the order of magnitude of the smallest length of the insert 1, it is possible to add a network of figure-eight shaped transverse openings 112, 124 in order to help the circulation of fluids. They are arranged so as to connect the openings 111, 121 of the first layer 11 and of the second layer 12 with each other. The desired number of interconnections Ni is for its part around 6 to 8 interconnections. The number of interconnections is defined as being the number of transverse openings 112, 124 extending from a single figure-eight shaped opening 111, 121 of the corresponding layer 11, 12. Too small a number of interconnections may lead to “congestion” creating an excess pressure in the openings 111, 121 of the first or the second layer 11, 12 and a flow rate in the transverse openings 112, 124 that is too large. Too large a number of transverse openings 112, 124 may increase, for its part, the risk of multiplication of “dead channels” by allowing the fluid the possibility of finding a strongly preferred trajectory.

When the layers of the insert comprise transverse openings 112, 124, the section of each opening 111, 121 of each layer 11, 12 is preferably greater than or equal to 0.7 mm² and less than or equal to 1.2 mm².

The plurality of pillars preferably comprises transverse openings at the lateral surface of the insert.

The surgical insert may further comprise a third layer 13 comprising figure-eight shaped openings 131 such as previously described and separated from the first layer 11 or from the second layer 12 by a plurality of spacers 15 such as previously described (FIG. 20).

In reality, the number of layers is not limited and the surgical insert may comprise 4, 5, 6, 7, 8, 9, or even 10 layers and more if needed.

The surgical insert may also comprise a mechanical reinforcement 16 (see FIG. 27). The mechanical reinforcement 16 takes up the flexion, torsion and compression forces so that the layers 11, 12 of the insert 1 do not experience deterioration. Further, the mechanical reinforcement 16 must not hinder the attachment of the insert 1.

The mechanical reinforcement 16 may be continuous or discontinuous. In the latter case, the mechanical reinforcement 16 is composed of a plurality of reinforcing units.

Preferably, the mechanical reinforcement 16 or each of the reinforcing units is an extra thickness of the surgical insert 1, in particular over one of the layers 11, 12 of the insert 1. This extra thickness is advantageously at least 1 mm. Further, the mechanical reinforcement 16 or each of the reinforcing units do not have figure-eight shaped openings.

In the case where an opening 161 intended to allow passage of an attachment screw (in particular round or oblong) is planned to pass through the mechanical reinforcement 16 or reinforcing unit, a border 162 at least 1 mm thick is preferably provided around said opening 161.

Such a mechanical reinforcement 16—the insert going into a bounding box BB with dimensions a, b, c of which the smallest dimension is c—is advantageous when at least one of the following conditions is satisfied:

$$\frac{a}{c} > 10 or \frac{b}{c} > 10 .$$

A bounding box BB for a set of points in three dimensions is defined as the parallelepiped box having the smallest volume and able to contain all of the points (see FIG. 27).

The surgical insert 1 may be used as a support in order to add at least one among particulating agents and active ingredients in order to avoid the risk of infection. Thus, the figure-eight openings 111, 121 may contain at least one among special agents and active ingredients.

Bacteriophages and phage lysines may be listed as special agents. Bacteriophages have the ability to target bacteria incorporated in a biofilm on the surface of the surgical insert. Phage lysines are hydrolytic enzymes.

The active ingredients may be in a liquid, hydrogel, cement, pulverulent or even microbead form. All these forms allow a prolonged release of active ingredients inhibiting bacterial adhesion, biofilm formation and bacterial proliferation.

Anti-infectious, antioncotic, and adhesion and bone healing activators may all be listed among active ingredients.

Antibiotics and antifungals may be listed among the anti-infectious agents.

Gentamicin, rifampicin (e.g. Rifadin®), ceftriaxone, quinolones such as fluoroquinolone (e.g. Ciprofloxacin®) and Moxifloxacin®), amoxicillin, tetracyclines (e.g. Doxycycline® and Minocycline®), mixtures of trimethoprim and sulfamethoxazole (e.g. Bactrim® and Eusaprim®), clindamycin, linezolid (e.g. Zyvoxyd®), fusidic acid, chlorhexidine and silver sulfadiazine may be listed among the antibiotics. The use of antibiotic impregnated surgical inserts makes it possible to locally release, e.g. at the site at risk of colonization, a high concentration of antibacterial agents. Antibiotic combinations may be advantageously used, such as rifampicin/tetracycline (in particular Minocycline®) and chlorhexidine/silver sulfadiazine.

Terbinafine, ketoconazole and amphotericin B may be listed among the antifungals.

The anti-osteosarcoma antioncotics may be listed among the antioncotics. Indeed, osteosarcoma is the most frequent of the malignant tumors taking birth in the bone. Examples of such antioncotics are methotrexate, doxorubicin, cyclophosphamide, cisplatin, ifosfamide and etoposide.

Growth factors and collagen, and also mesenchymatous stem cells may be listed among the activators of bone adhesion and healing.

Other biological materials may also be added to the surgical insert.

According to a second aspect, the present invention also covers a bone cement comprising:
a pulverulent solid phase comprising polysaccharides that are natural or derived from natural compounds and a ceramic filler;
a liquid acrylate; and
a polymerizer.

It may further comprise at least one among:
a radio-opacifier; and
a swelling-limiting agent.

The natural polysaccharide or derivative of natural compounds may be selected among a derived from cellulose (for example cellulose acetate) and a starch (for example wheat starch, potato starch, corn starch and pea starch), and also mixtures thereof. Preferably, the polysaccharide that is natural or derived from natural compounds may be a mixture of cellulose acetate and starch, in particular potato, pea or corn starch. Potato starch further has the advantage of being easily metabolized by the human or animal body.

The pulverulent solid phase may further comprise alpha amylase, more specifically when it comprises a starch other than potato starch.

Cellulose acetate is a cellulose derivative. This latter is a carbohydrate made up of a linear chain of D-glucose molecules. It is a biopolymer which makes up the wall of vegetable cells and represents 35 to 50% of the vegetable biomass. Cellulose acetate thus differs from cellulose by the fact that the hydroxy functional groups of the D-glucose molecule are replaced by acetate functional groups.

The proportion by mass of cellulose acetate relative to the total mass of the bone substitute may be included between 6% and 24%, preferably between 8% and 20%, still preferably between 10% and 17%, for example about 15%.

Starch is a storage molecule of higher plants, meaning all plants excluding algae and mosses. Starch is found principally in seeds, for example grain seeds like corn, wheat and rice. Starch may also be found in roots, tubers (for example the potato tuber) and fruits. Starch is a complex carbohydrate composed of D-glucose units. More specifically, starch is a mixture of two homopolymers composed of D-glucose, amylose and amylopectin units, so that it belongs to the polysaccharide family, more specifically the natural polysaccharides, meaning that it is found available directly in nature.

The proportion by mass of starch, in particular potato, pea or corn relative to the total mass of the bone substitute may be included between 5% and 30%, preferably between 5% and 20%, still preferably between 5% and 10%, for example about 7%.

The ceramic filler may be selected among alumina, calcium sulfate hemihydrate, hydroxyapatite, tricalcium phosphate, bioglass and mixtures thereof. The use of the ceramic filler is considered for economic reasons but also for the properties that this ceramic filler may contribute to the bone cement, in particular properties of mechanical strength under compression and biological properties in order to improve the bioactivity and bone regrowth.

The proportion by mass of the ceramic filler may be included between 18% and 45% relative to the total mass of the bone substitute.

The acrylate may be hydroxyethylmethacrylate (HEMA), methylmethacrylate (MMA) or a mixture thereof.

HEMA is a colorless liquid organic compound which has the property of rapidly polymerizing. It is a monomer which is used in fabricating many polymers, like for example polyhydroxyethylmethacrylate (pHEMA). pHEMA is a hydrophobic polymer with hydrogel properties. It has the ability to swell in the presence of water due to the hydrophilic part of the HEMA without leading to the dissolution of the polymer network. In the literature, cases of hydrogels are reported where the mass is multiplied by a thousand by absorption of water.

The proportion by mass of acrylate relative to the total mass of the bone substitute may be included between 30% and 50%, preferably between 35% and 45%, preferably between 37.5% and 42.5%, for example 40%.

The polymerizer is a reagent making it possible to initiate the polymerization of the monomers with each other in the reaction medium. The polymerizer may be a radical polymerizer. A radical polymerizer is a compound capable under certain conditions of generating free radicals which serve to initiate the polymerization of the acrylate monomers in order to form a polyacrylate such as polyhydroxyethylmethacrylate, polymethylmethacrylate or a polymer obtained from HEMA and MMA.

This radical polymerizer may be a chemical initiator or photochemical initiator.

A thermal initiator is a compound generating free radicals by thermal decomposition. Benzoyl peroxide (BPO) is an example of a chemical initiator. In that case, a catalyst may be used, for example dimethyl-p-toluidine (DMT).

A photochemical initiator is a compound generating free radicals under the effect of radiation emitted by a radiation source. When they are exposed to radiation, they enter an excited state and release free radicals. Photopolymerization is particularly suited to shaping the cement by 3D printing. The radiation may in particular be ultraviolet radiation. An example of a photochemical initiator is the riboflavin/triethanolamine system.

A radio-opacifier is a compound whose function is to increase the radio-opacity of a material. Radio-opacity is defined as the opacity to X-rays and radio frequency waves, meaning the relative inability of these kinds of radiation to pass through this material. Radio-opacifiers are principally used in the medical domain, because they may be used for example as contrast agents for radiography. Medical devices, such as implants, often contain it for improving visualization during implantation of temporary devices such as catheters or placement of permanent implants. Metal implants generally have an inherent radio-opacity whereas polymer-based implants need addition of a radio-opacifier for increasing the contrast thereof.

The radio-opacifier may be selected from zirconium dioxide (ZrO₂), barium sulfate (BaSO₄) and mixtures thereof.

Since bone cement is in constant contact with human body fluids, it may be necessary to add an agent limiting the swelling of pHEMA. In fact, if the swelling of the pHEMA becomes too great, the bone cement may exert a stress on the zone in which it is implanted and cause lesions or pain. This agent limiting the swelling may be water or an aqueous solution comprising a medication.

The bone cement may further comprise a setting activator and/or retarder.

A setting activator and a setting retarder are adjuvants used for accelerating or slowing the setting of the bone cement. It thus makes it possible to respectively reduce or increase the time during which the cement can be modeled. Depending on how the bone cement will be used, it may sometimes be necessary to increase the setting time thereof to give the practitioner more time to model it and give it the intended complex shape whereas in other uses, it may be necessary to reduce the setting time. Thus, for example, if the clinical pathology is simple, rapid setting serves to reduce the perioperative time whereas in contrast, for complex procedures, the setting time may be increased. In another context, if the cement is used for 3D printing, the setting time must be sufficiently long so that the machine can print the 3D part.

Hydroquinone is an example of a setting activator. Vitamin C is an example of a setting retarder. Additionally, vitamin C may act as a setting activator depending on the quantity of product added. More specifically, when the vitamin C concentration is low, it has an activator effect instead. When the vitamin C concentration is high, it has instead a delaying effect.

The bone substitute may preferably have a Young's modulus included between 1 MPa and 32 MPa after seven days of immersion in phosphate-buffered saline, preferably over 5 MPa.

The bone substitute preferably has a shearing modulus included between 100 Pa·s and 150,000 Pa·s, preferably included between 2000 Pa·s and 20,000 Pa·s.

### EXAMPLES

### Example 1: Influence of the Quantities of the Solid Phase Components on the Properties of the Cement

In this example, the Young's modulus, the complex shearing modulus (G*) and the swelling are going to be determined as a function of the proportion of various solid phase components, the ceramic filler, starch and cellulose acetate.

The proportion by mass of HEMA was set at 40% by mass and the proportions by mass of ceramic filler, cellulose acetate and starch relative to the total mass of the bone cement vary within the following ranges:
18% to 45% by mass of ceramic filler,
6 to 30% by mass of starch, and
6 to 24% by mass of cellulose acetate.

### Preparation of the Cement:

In order to prepare the cement, the liquid and solid phases are initially separated. They are then brought into contact and mixed to get a homogeneous paste. The paste is then shaped in different ways depending on the intended shape of the cement, for example by molding, casting or even by 3D printing. Placing the powder in contact with the liquid phase leads to the polymerization of the HEMA. This polymerization reaction is an exothermal reaction. Once the cement hardens, it may for example be withdrawn from the mold, if the shaping was done by casting.

In order to study the variation of properties of the bone cement when the proportion by mass of ceramic filler, cellulose acetate and starch vary, X₁ is defined as being the ratio between the proportion by mass of ceramic filler relative to the total mass of the solid phase, meaning the mass ratio of ceramic filler in the solid phase, X₂ as being the ratio between the proportion by mass of starch relative to the total mass of the solid phase, meaning the mass ratio of starch in the solid phase, and X₃ as being the ratio between the proportion by mass of cellulose acetate relative to the total mass of the solid phase, meaning the mass ratio of cellulose acetate in the solid phase. The upper and lower boundaries of each mass ratio are indicated in Table 3.

TABLE 3

| Mass ratio | X1 | X2 | X3 |
|---|---|---|---|
| Lower bound | 0.3 | 0.1 | 0.1 |
| Upper bound | 0.75 | 0.5 | 0.4 |

FIG. 29 shows the ternary diagram for variation of X₁, X₂ and X₃. The surrounded zone is the domain which will be studied.

### Measurement of the Young's Modulus

In a first step, the Young's modulus of the cement after setting is measured in the study domain. The Young's modulus is measured by a compression test according to the ISO 5833:2002 standard. The test sample is a cylindrical sample 10 mm in diameter and 15 mm high. The sample was previously immersed for seven days in a phosphate-buffered saline solution (PBS).

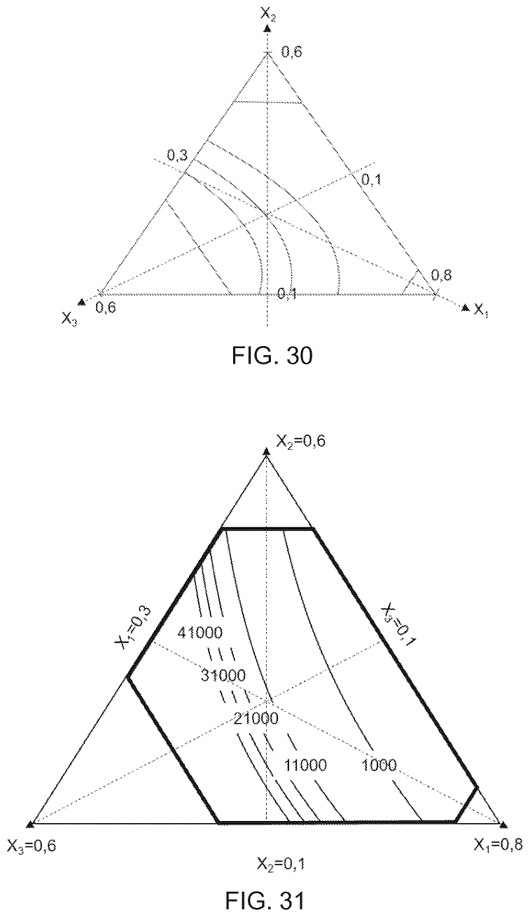

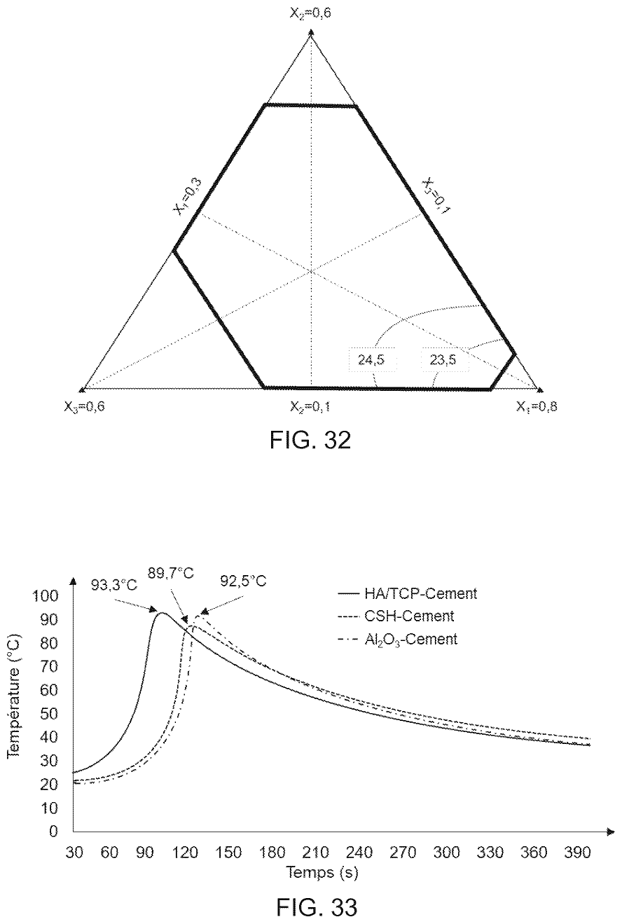

FIG. 30 shows the change of the Young's modulus in the domain of study. Note that the higher the cellulose acetate ratio is raised, the more the Young's modulus increases. A minimum Young's modulus of 1 MPa and a maximum Young's modulus of 32 MPa are measured.

Thus, the Young's modulus varies widely over the study domain. The Young's modulus of the cement must be close to that of the bone into which the cement will be injected. This large range thus serves to adapt the Young's modulus of the cement to the zone where the cement is injected.

### Measurement of the Shearing Modulus

In a second step, the shearing modulus of the cement after setting is measured in the study domain. The shearing modulus is measured in a rheometer in controlled-speed mode. The measurement was done at 25° C. with a 40 mm diameter and a 2° cone angle in the shearing-rate range, with a reading every 10 s for 300 s.

FIG. 31 shows the change of the shearing modulus in the domain of study. Note that, as for the Young's modulus, the shearing modulus increases when the cellulose acetate mass ratio increases. A minimum of 100 Pa·s and a maximum of 150,000 Pa·s are measured.

Thus, the shearing modulus varies widely over the study domain. The shearing modulus of the cement must be particularly controlled for allowing an easy injection, but such that the cement is not to liquid so as to avoid leaks or clinical complications. This large range thus serves to adapt the shearing modulus of the cement to the injection zone of the cement and especially to the specifics required by the clinician (either in pasty form to be applied with a spatula, or in more liquid form to be injected).

### Measurement of the Swelling

In a third step, the swelling of the cement after setting is measured in the study domain. The swelling is measured by taking the ratio between the volume of a cylindrical sample 10 mm in diameter and 15 mm high before immersion and after seven days immersed in a PBS solution.

FIG. 24 shows the change of the swelling in the domain of study. Note that the swelling decreases when the mass ratio of the ceramic filler increases. A minimum of 21% and a maximum of 32% are measured.

The swelling may thus be adapted to the injection zone of the cement. The swelling must be sufficiently large to fully conform to the zone which should be protected but not so large that the cement applies stresses on the bones. Too much swelling may in fact damage the bone and lead to complications.

### Example 2: Influence of the Type of the Ceramic Filler on the Properties of the Cement

In this example, the nature of the ceramic filler in the solid phase of the bone cement is modified in order to study the influence of the nature of the ceramic filler on the properties cement.

To evaluate the preparation of the cement, three formulations were made according to Table 4 below which gives the proportions by mass:

TABLE 4

| | Example 2.1 | Example 2.2 | Example 2.3 |
|---|---|---|---|
| Liquid | DMT (0.07%) | DMT (0.07%) | DMT (0.07%) |
| phase | HEMA (39.93%) | HEMA (39.93%) | HEMA (39.93%) |
| Solid | cellulose | cellulose | cellulose |
| phase | acetate (12.86%) | acetate (13.82%) | acetate (14.93%) |
| (apart | starch (6.01%) | starch (6.02%) | starch (6.5%) |
| from | BPO (0.22%) | BPO (0.22%) | BPO (0.22%) |
| ceramic | | | BPO |
| filler) | | | |
| Ceramic | alumina (40.91%) | CSH (39.94%) | HA (19.17%) |
| filler | | | TCP (19.17%) |

CSH: calcium sulfate hemihydrate
HA: hydroxyapatite
TCP: tricalcium phosphate

These three formulations were shaped using a cylindrical silicon mold 10 mm in diameter and 15 mm high. They are unmolded after the cement hardened.

The acrylic cements are characterized by the setting time and by the maximum polymerization temperature. The setting time corresponds to the time necessary for the average setting temperature to be reached. The average temperature is represented by the average of the maximum polymerization temperature and the ambient temperature of the part.

FIG. 33 shows the change of temperature with time for the three examples. The measurements are done with a T-type thermocouple and are shown in Table 5.

TABLE 5

| Ceramic filler | Alumina | CSH | HA/TCP |
|---|---|---|---|
| Setting time (min) | 1.95 | 1.87 | 1.47 |
| Maximum | 92.5 ± 2 | 89.7 ± 2 | 93.3 ± 2 |
| temperature (°C) | | | |

Thus, the setting temperature remains relatively constant as a function of the ceramic used.

As a function of the ceramic filler, the setting time varies from 90 to 120 seconds. It is however possible to increase it by keeping the product in a cold room, the setting time then goes to 300 seconds. The addition of setting retardant may also be considered depending on the application and the shaping of the cement.

Vitamin C is known for its properties both retarding setting and activating setting on acrylic cements like polymethylmethacrylate (PMMA). Thus, depending on the cement composition, it may both have a retarding property and an activating property or both at the same time. The setting retardant effect was tested on example 2.1.

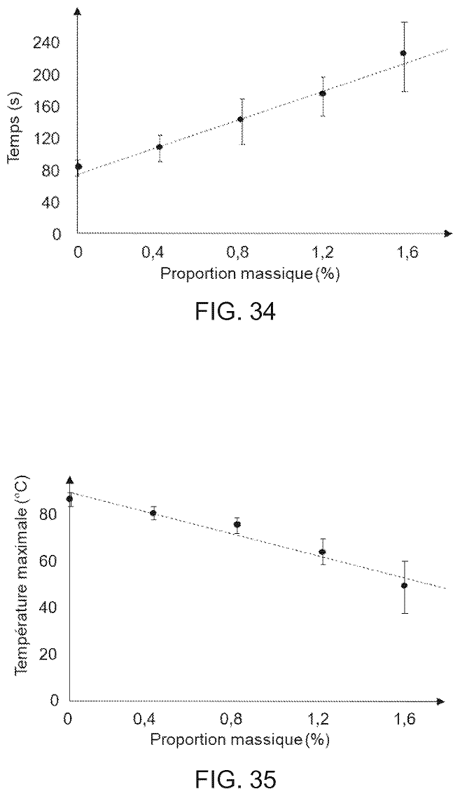

FIG. 34 shows the change of the setting time as a function of the mass proportion of vitamin C and FIG. 35 shows the change of the maximum temperature as a function of the mass proportion of vitamin C. Note that with the formulation without vitamin C, the setting time is 83 s and the maximum temperature is 86.7° C. With a mass proportion of vitamin of 1.6%, the maximum proportion tested, the setting time is 225 s and the maximum temperature is 48.9° C.

The quantity of BPO and DMT also has an influence on the setting time of the cement. The larger the quantity of BPO-DMT, the longer the setting time is as well. Thus, a setting time of 5 min, for 0.3 mL of BPO-DMT and a setting time of 40 min, for 0.7 mL of BPO-DMT are observed. However, increasing the quantity of BPO-DMT can result in an increase of the cytotoxicity of the cement. The compromise between cytotoxicity, a subject which will be discussed below, and setting time is found for a quantity of 0.5 mL of BPO-DMT and a setting time of 12 min.

The Young's modulus and the stress at rupture of the cement after setting was measured by a compression test for example 2.3, filled with HA/TCP. The cement had a cylindrical shape 15 mm high and 10 mm in diameter. The Young's modulus was measured before and after immersion for seven days in PBS. The results are given in Table 6.

TABLE 6

| | | Cement after |
|---|---|---|
| | | 7 days of |
| Cement | Dry Cement | immersion |
| Young's modulus | 6139.5 ± 707.8 | 10.4 ± 1.08 |
| (MPa) | | |
| Stress at break | 62.49 ± 3.84 | 0.57 ± 0.4 |
| (MPa) | | |

The sample after immersion showed a very ductile behavior; it cut easily with a scalpel, and had a porous volume of about 30%.

To evaluate the behavior of the cement in a liquid medium, a hydrostatic weighing device is used. A hydrostatic weighing device serves to measure the weight of an object of indeterminable apparent volume such as pulverulent materials. The device thus serves to measure the swelling and also the open porosity of the cement. The measurement method consists of weighing the dried cement pellets, of cylindrical shape with a 10 mm diameter and a 2 mm height, and then immersing them in a liquid medium at a rate of 15 mL per pellet under stirring at 180 RPM and ambient temperature. On each sampling day, the samples are weighed wet and in the basket of the hydrostatic balance, underwater. Each weighing is repeated three times and on five identical pellets.

The swelling of the material is calculated with the formula Math 10 and the porosity with the formula Math 11.

$$Swelling = \frac{{M}_{h} - {M}_{s}}{{M}_{s}} \cdot 100 \qquad [ Math . 10 ]$$

$$PO = \frac{{M}_{h} - {M}_{s}}{{M}_{h} - {M}_{e}} \cdot 100 \qquad [ Math . 11 ]$$

where Mₛ is the dry mass of the pellet, Mₕ is the wet mass of the pellet, PO is the open porosity of the cement pellet, and Mₑ is the mass underwater of the pellet.

The swelling and the open porosity of the three cement formulations were tested in different liquid media:
distilled water,
PBS,
simulated body fluid (SBF), and
alcohol.

The swelling of the pellets is essentially due to the pHEMA. This swelling is of interest in connection with an implantation in a boney site. It serves to fill a boney gap whereas the shrinkage of PMMA-based cements for example favors the harmful development of fibrous tissue at the cement-bone interface. It also serves to deal with the possibility, from the first hours, of an incomplete filling of the bone cavity during placement by the surgeon.

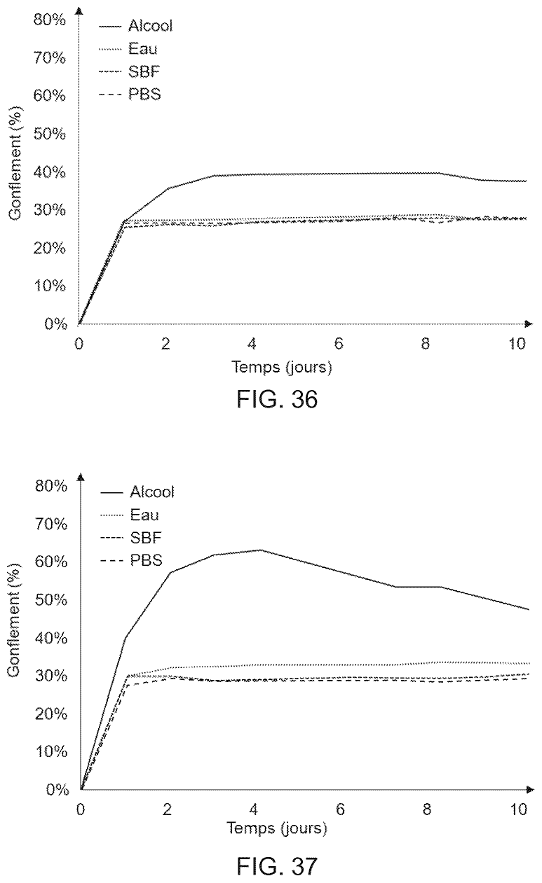

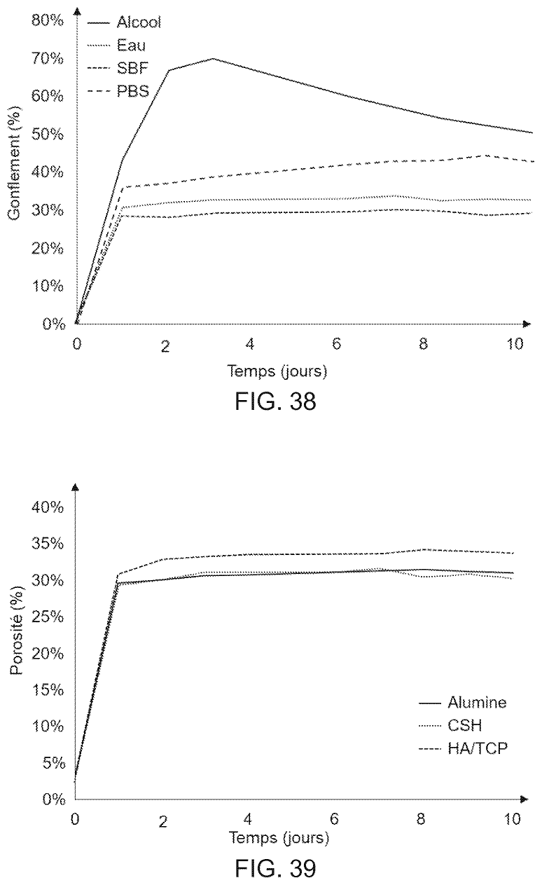

FIGS. 36, 37 and 38 show the change of the swelling of the formulation 1, 2 and 3 respectively in the various liquid media as a function of the number of days of immersion. The swelling happens very quickly, at the end of one day and levels off in the vicinity for the aqueous media, meaning water, PBS and SBF. It is observed that the swelling in alcohol is significantly greater than in aqueous media, because the maximum swelling of the cement reaches 40% on the third day. Despite the very large swelling in alcohol, which can reach up to 60%, no breakdown of the pellet took place.

FIG. 39 shows the change of the open porosity from formulations 1, 2 and 3 as a function of the number of days of immersion in distilled water. Before immersion, the samples have an open porosity of 3%. In one day, a very rapid increase of the open porosity is observed before leveling off at 30% for the formulations 1 and 2 and 33% for the formulation 3. This difference in open porosity between the formulations is to be connected with the swelling of the formulations in water from FIGS. 16, 17 and 18. It can therefore be deduced from this that in the case of water the swelling is due to the open porosity. Further, after reaching the ceiling of the open porosity, it is seen that the swelling of the cement no longer increases. It can be assumed that the water does not penetrate or only penetrates very little into the cement or that the closed porosity of the cement is small or even nearly nonexistent.

The swelling may be limited by pre-saturating the cement with an aqueous fluid. Indeed, getting a swelling of the cement because of hydration is certainly very important for the reasons previously mentioned. However, in many pathologies involved with bone filling, the bone may already be fragile (e.g. osteoporosis, osteosarcoma). Incorporating a cement which would excessively fill up with biological fluid could make the surrounding bones fragile and lead to other bone disorders.

Distilled water was added to the liquid phase before mixing the two phases. Tests as to the mixability and the setting of the cement led us to set the quantity at 0.28 g of water for 1.3 g of HEMA. In fact, incorporating water in the cement does not improve the mixability. The addition of 0.28 g makes it possible to go from a cement without water which swells 34% at the end of two days to a cement which swells 24% at the end of two days.

The injectability criterion is important for the administration of the cement, either by injecting by the clinician in the bone for example by vertebroplasty, by kyphoplasty or by deposit by spatula, or by depositing the cement on a support manually or better by rapid prototyping.

Rapid prototyping is a computer-controlled fabrication method. It makes it possible to quickly implement models which will be produced in order to reduce the development time. The tools necessary for setting up this method are low-cost while guaranteeing excellent performance of the final product. Finally, by the use of machines like 3D printers and machining, rapid prototyping is capable of implementing extremely complex shapes, particularly suited for example, to bone implants.

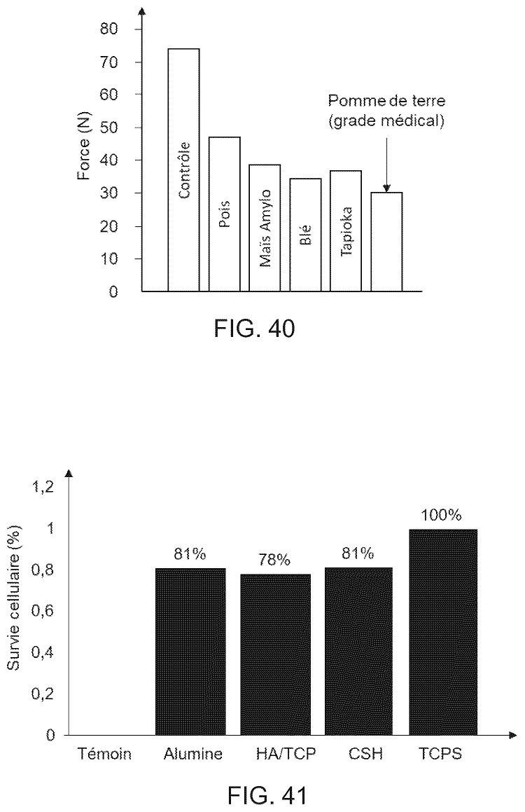

For a single quantity of starch, the injectability force was measured for six botanical varieties of starch. The results are shown in FIG. 40. The injectability test consists of placing 5 mL of cement in a sterile 10 ml syringe, next placing the syringe in a compression machine, and then measuring the force applied for constant displacement of 1 mm/s.

It is important to evaluate the cytotoxicity of the cement, meaning, the capacity thereof to attack or even destroy the cells of the organism. To do that, the pellets which were used to test the swelling and open porosity of the cement are rinsed and dried. They are then sterilized by UV radiation for 30 minutes per surface.

The cement pellets are placed in a 24-well plate and covered by 1 mL of Eagle minimum essential medium type culture medium containing bovine proteins. The plate is incubated at 37° C. for 48 hours. At the end of two days, the culture medium in contact with the pellets is sampled and 100 μL is placed on MC3T3-E1 osteoblasts with 60% confluence. The survival rate is determined by measuring the cellular activity using the Alamar blue method.

FIG. 41 shows the cellular survival rate as a function of each cement formulation and of two controls, a first based on 100% alumina, and the second based on tissue culture polystyrene (TCPS), material constituting the culture plates. It is observed that the alumina-based control leads to the death of all the cells whereas the pellets from the three formulations of the cement have a cellular survival rate with a minimum of 78% for formulation 3 and a maximum of 81% for the formulations 1 and 2.

In order to reduce the cytotoxicity of the cement formulations, it is necessary to divide the quantity of BPO and DMT by three, meaning, to shift to 19 mL and to follow the following protocol:
wash the cement with ether three times for 20 min.,
wash the cement with ethanol for 30 min.,
wash the cement with water for 48 hours, and
dry the cement in the oven.

An increase of the cellular survival rate is observed when the preceding protocol is followed, with a 92% cellular survival rate for the three formulations.

Because of the hydrogel properties thereof, the bone cement may be used as a substance release agent in the body, in particular medication. In order to study in vitro the substance release quantity, the pellets underwent a desorption test and an impregnation test with ciprofloxacin (CFX).

CFX belongs to the family of fluoroquinolones. This active ingredient is used in the treatment of many infections including those of the skin and soft tissues, but also osteoarticular infections. It is an antibiotic whose action serves to inhibit topoisomerase I and topoisomerase IV which are responsible for replication, transcription, repair and recombination of bacterial DNA.

The desorption test was done according to the following protocol:
the cement pellets are left to be impregnated 24 hours in a solution of CFX for perfusion at a concentration of 2 g/L under stirring at 180 RPM and ambient temperature,
the cement pellets are added to a volume of 20 mL of sodium hydroxide at 0.1 mol/L and placed under stirring at 80 RPM at 37° C. for 12 hours, and
the quantity of CFX released by the cement pellets is measured by UV/visible spectrophotometry.

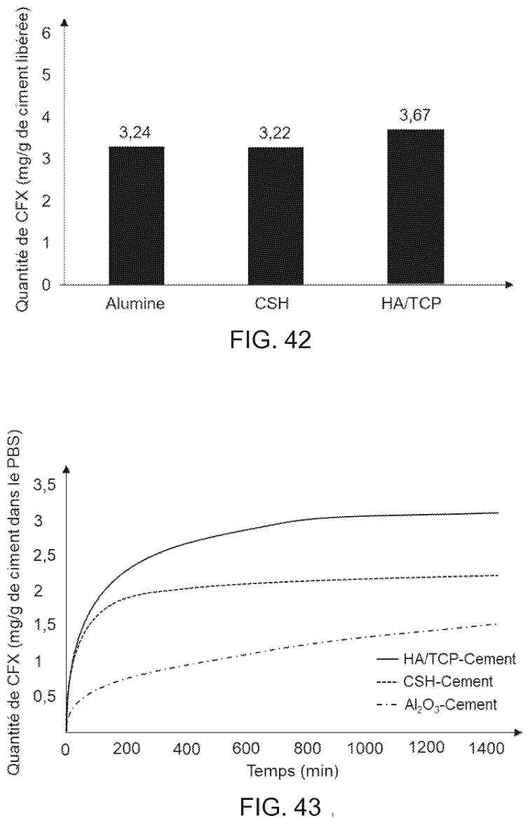

FIG. 42 shows the total quantities of antibiotic present on the various cements. During the impregnation, the cements absorbed similar quantities of CFX, around 3.2 mg per gram of cement, seemingly without showing a significant difference according to the filler.

The impregnation test was done according to the following protocol:
the cement pellets are left to be impregnated 24 hours in a solution of ciprofloxacin for perfusion at a concentration of 2 g/L under stirring at 180 RPM and ambient temperature,
the cement pellets are dried for 24 hours at 37° C.,
the pellets are placed in a release cell comprising a ruby bead 5 mm in diameter, more specifically on a bed of glass beads 1 mm in diameter,
a flow of PBS coming from a 100 mL volume reservoir is sent via a pump into a UV/visible spectrophotometer, then into the release cell, then returned to the reservoir and again passed in the UV/visible spectrophotometer.

The UV/visible spectrophotometer measures the absorbance at 270 nm every five minutes during the first six hours and then at 15-minute intervals up to 24 hours.

FIG. 43 shows the ciprofloxacin release profile as a function of time for the three cement formulations. For the cement filled with alumina, formulation 1, the release profile seems extended. Even if the release is faster during the first hours (0.50 mg per gram of cement at one hour), the plateau of the maximum concentration does not seem totally reached at 24 hours: the quantity released is then 1.53 mg per gram of cement and that represents 47% of the quantity released by the desorption test. One may therefore assume that the release continues after 24 hours.

For the cement filled with CSH, formulation 2, the release of the CFX occurs during the first hours, because the plateau is reached at 15 hours at 2.2 mg of CFX per gram of cement, which represents 69% of its total quantity of CFX.

The biphasic cement filled with HA/TCP, formulation 3, is the cement which released the largest quantity of antibiotic and the most quickly. The active ingredient is released progressively and reaches a quantity released at 24 hours of 3.1 mg of CFX per gram of cement or 86% of the total quantity present on the cement. From this it can be concluded that the biphasic cement has good results for a massive release of CFX, because it releases the active ingredient in large quantity and gives the best release rate. Knowing that the release by a cement is at the local scale, therefore in special medium, the release of this antibiotic by such cements is clinically conceivable.

Further, it would also be conceivable to adapt the concentration of the impregnation solution in order to increase the quantity absorbed by the cement. The form of the active ingredient may also vary the release profile thereof.

For prophylactic antibiotics release, the cement filled with alumina is an attractive solution, as is the CSH. An extended release profile may be attractive for the release of medications such as, like here, antibiotics, but also anticancer agents or analgesics, for example.

### Example 3: Optimal Composition of the Cement for Deposit on a Titanium Insert

In this example, an optimal composition of the bone cement will be presented. This optimal composition is the best compromise between the various properties of the bone cement.

The optimal composition of the bone cement comprises:
a liquid phase composed of 1.8 g of HEMA and 0.0031 g of DMT, and
a solid phase composed of 1.733 g of HA/TCP, 0.675 g of cellulose acetate, 0.294 g of starch and 0.01 g of BPO.

The two phases are brought into contact in order to form a paste which is cast in a silicone mold. After setting of the cement, it is unmolded. Four cement pellets are made.

The four cement pellets are characterized after seven days of immersion in PBS. The characterization results are shown in the following table.

TABLE 7

| | E (MPa) | G* (Pa) | Swelling (%) |
|---|---|---|---|
| Optimal conditions | >5 | 2000 to 20,000 | <24.5 |
| Average with 95% | 7.3 ± 3.3 | 12,640 ± 1,765 | 24.4 ± 0.5 |
| confidence interval | | | |

This composition is the most optimized, meaning that it is the best compromise between the mechanical properties, injectability and a moderate swelling.

### Example 4: Two-Layer Surgical Insert without Spacers

In this example, a two-layer surgical insert 1 will be presented with two zones 122, 123 of different porosities in the second layer 12.

A surgical insert 1 was implemented by rapid prototyping. FIG. 9 shows the two faces of the insert 1 and the properties thereof are shown in the table below.

TABLE 8

| Properties | Values |
|---|---|
| Total volume | 338.96 |
| | mm³ |
| Porosity of the first zone 122 of the second | 0.431 |
| layer 12 | |
| Porosity of the second zone 123 of the | 0.397 |
| second layer 12 | |
| Porosity of the first layer 11 | 0.728 |
| Growth ratio between the openings of the | 1.82 |
| second zone and those of the first zone | |
| Overall porosity | 0.487 |
| Estimated maximum stress | 22.6 |
| | MPa |

The openings have the dimensions shown in the following table.

TABLE 9

| | | | | First | Second |
|---|---|---|---|---|---|
| | Maxi- | Maxi- | Mini- | radius | radius |
| | mum | mum | mum | of | of |
| | length | width | width | curvature | curvature |
| Part | (mm) | (mm) | (mm) | (mm) | (mm) |
| Second | 4.92 | 2.49 | 1.64 | 1.22 | 0.82 |
| layer 12, | | | | | |
| first | | | | | |
| zone 122 | | | | | |
| Second | 2.70 | 1.37 | 0.90 | 0.67 | 0.45 |
| layer 12, | | | | | |
| second | | | | | |
| zone | | | | | |
| 123 | | | | | |
| First layer | 8.01 | 4.05 | 2.67 | 1.99 | 1.34 |
| 11 | | | | | |

A deposit of 4 mm of the bone cement presented in example 1 was placed on the surgical insert 1. The covered insert was immersed in PBS for four days. The properties of the insert 1 with the deposit after immersion are shown in the table below.

TABLE 10

| Properties | Values |
|---|---|
| Total volume | 514.13 |
| | mm³ |
| Porosity of the first zone 122 | 0.100 |
| of the second layer 12 | |
| Porosity of the second zone | 0.040 |
| 123 of the second layer 12 | |
| Porosity of the first layer 11 | 0.481 |
| Overall porosity | 0.290 |

### Example 5: Surgical Insert with Mechanical Reinforcement

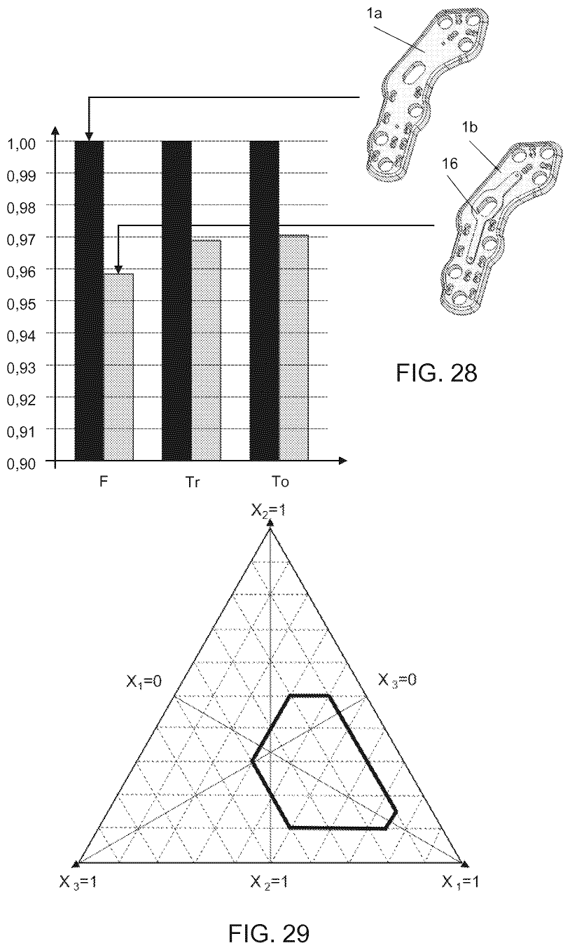

FIG. 28 shows the results of the simulation of two inserts both having figure-eight shaped openings. One 1a does not have mechanical reinforcement (black bars) and the other 1b has a mechanical reinforcement 16 (gray bars). The bars represent the maximum simulated movement in the case where the insert is subject to a flexion F, traction Tr or torsion To force. The values are normalized to the values obtained for the insert 1a without mechanical reinforcement. Note that the maximum displacement is less during the application of the three types of force for the insert 1b with mechanical reinforcement compared to the one without mechanical reinforcement.

### REFERENCE NUMBERS

1: surgical insert,
11: first layer,
111: openings in the first layer,
112: transverse openings in the first layer,
12: second layer,
121: openings in the second layer,
122: first zone of the second layer,
1221: openings in the first zone of the second layer,
123: second zone of the second layer,
1231: openings in the second zone of the second layer,
124: transverse openings in the second layer,
13: third layer,
131: openings in the third layer,
14: attachment tabs,
15: plurality of spacers, and
151: openings in the plurality of spacers.

1-32. (canceled)

33. A bone cement comprising:
a pulverulent solid phase comprising polysaccharides that are natural or derived from natural compounds and a ceramic filler;
a liquid acrylate; and
a polymerizer.

34. The bone cement according to claim 33, further comprising at least one from:
a radio-opacifier; and
a swelling-limiting agent.

35. The bone cement according to claim 33, further comprising a setting activator or retarder, where, in particular, said setting activator or retarder is vitamin C.

36. The bone cement according to claim 33, wherein the proportion by mass of the acrylate relative to the total mass of the bone substitute is included between 30% and 50%, preferably between 35% and 45%, preferably between 37.5% and 42.5%, for example 40%.

37. The bone cement according to claim 33, having a Young's modulus included between 1 MPa and 32 MPa after seven days of immersion in phosphate-buffered saline, preferably over 5 MPa.

38. The bone cement according to claim 33 having a shearing modulus included between 100 Pa·s and 150,000 Pa·s, preferably included between 2000 Pa·s and 20,000 Pa·s.

39. The bone cement according to 33 having a swelling ratio included between 21% and 32% after seven days of immersion in phosphate-buffered saline.

40. A surgical insert in a bone cement and comprising:
a first layer, and
a second layer,
wherein the first layer and the second layer comprise openings having a figure-eight shape;
wherein the bone cement comprises:
a pulverulent solid phase comprising polysaccharides that are natural or derived from natural compounds and a ceramic filler;
a liquid acrylate; and
a polymerizer.

41. The surgical insert according to claim 40, wherein the openings are laid out according to a pattern selected from a square pattern, a hexagonal pattern and a heterogeneous pattern.

42. The surgical insert according to claim 40, wherein the arrangement of the figure-eight shaped openings on the second layer result from a homothetic duplication of the arrangement of the figure-eight shaped openings on the first layer.

43. The surgical insert according to claim 40, wherein first layer has a porosity greater than that of the second layer.

44. The surgical insert according to claim 40, wherein the second layer has an orientation guide.

45. The surgical insert according to claim 40, further comprising a plurality of spacers placed between the first layer and the second layer.

46. The surgical insert according to claim 45, wherein the thickness h of each layer must satisfy the relationship:
$$h > \frac{min \{ l , L \}}{4} \qquad [ Math . 1 ]$$
where l is the maximum width of the surgical insert and L is the maximum length of the surgical insert.

47. The surgical insert according to claim 45, wherein each among the plurality of spacers is a pillar whose transverse section has a figure-eight shape.

48. The surgical insert according to claim 45, wherein each among the plurality of pillars is helical and has a torsion angle over 0° and less than or equal to 180°.

49. The surgical insert according to claim 45, wherein each among the plurality of pillars comprises transverse openings at the lateral surface thereof.

50. The surgical insert according to claim 40, wherein, at least one of the openings of the first layer and the openings of the second layer have a lateral wall formed in the thickness of the corresponding layer, and wherein at least one of the first layer and the second layer comprise figure-eight shaped transverse openings and extending between the lateral wall of two openings of the corresponding layer.

51. The surgical insert according to claim 40, further comprising an attachment tab extending from one among the first layers.

52. The surgical insert according to claim 40, further comprising a mechanical reinforcement.
